(19) United States
(12) Patent Application Publication
(10) Pub. No.: US 2021/0381131 A1
(43) Pub. Date: Dec. 9, 2021

(54) Hollow fibers for outside-in-dialysis applications

(71) Applicant: Novaflux Inc., Princeton, NJ (US)

(72) Inventors: Ilaria Geremia, Teglio Veneto (IT); Dimitrios Stamatialis, Deventer (NL); Mohamed Labib, West Palm Beach, FL (US); Peter Materna Materna, Metuchen, NJ (US)

(21) Appl. No.: 17/339,116

(22) Filed: Jun. 4, 2021

Related U.S. Application Data
(60) Provisional application No. 63/034,790, filed on Jun. 4, 2020.

(51) Int. Cl. D01F 8/16; A61M 1/16; D01D 5/06; D01D 5/24

(52) U.S. Cl. CPC D01F 8/16; A61M 1/1625; D01D 5/06; D01D 5/24; D10B 2509/00; A61M 2205/7527; D10B 2331/06; D10B 2401/022; D10B 2401/10; A61M 2202/0421

(57) ABSTRACT

Disclosed are hollow fibers suitable for use in dialysis in an outside-in configuration. For such fibers, it is desirable that the fiber have a low albumin sieving coefficient and have a permeability high enough to be considered a High Flux dialyzer, and it is desirable that the outer (blood-facing) surface have a sufficiently small roughness and be hydrophilic. It is desirable that there be a selective layer on the outer surface and, interiorly of that, a porous structurally supportive region, which may contain elongated macrovoids. Such a fiber may be spun through a triple-concentric spinneret that produces a bore liquid surrounded by dope surrounded by a shower. The shower and the coagulation bath may be pure water, which is a non-solvent. The process may be performed at room temperature. Spinning parameters are discussed.

### CROSS-REFERENCE TO RELATED APPLICATIONS

This application claims priority to U.S. Provisional Application Ser. No. 63/034,790, that was filed with the United States Patent and Trademark Office on Jun. 4, 2020. The entire disclosure of U.S. Provisional Application Ser. No. 63/034,790 is incorporated herein by reference.

### BACKGROUND OF THE INVENTION

Porous-walled hollow fiber membranes are widely used for separation and filtration applications such as dialysis. Usually such fibers are manufactured by extruding or spinning polymeric material through a spinneret, while using a phase separation technique that results in the desired porosity. In dialysis, such hollow fibers are almost always used with blood flowing through the lumen of the fiber while a dialysis solution flows on the outside of the fiber so as to remove uremic toxins from patient's blood during hemodialysis or related therapies. This is termed the inside-out configuration. There is another flow configuration that is not used in hemodialysis, referred to as the outside-in configuration, where blood flows on the outside of the fiber and dialysate flows in the lumen. The outside-in configuration has certain advantages compared to the standard inside-out configuration. For example, a dialyzer operated in the outside-in configuration may be less affected by the possible formation of blood clots, and thus may be more suitable for long-duration or continuous use. Thus, there remains a need for a hollow fiber that can perform hemodialysis in the outside-in configuration. Such a fiber should have an external surface that is hemocompatible, which is being hydrophilic and having a sufficiently small surface roughness of the blood-facing external surface. The ideal outside-in hollow fiber should have high flux properties or a high coefficient of ultrafiltration (KUF) as to allow for passage of water and small and middle molecular weight uremic toxins through the fiber wall, or in other words such fiber should achieve both diffusive and convective clearance of uremic toxins. At the same time, the ideal fiber should result in very little loss of albumin from the blood during dialysis. These two goals of high KUF and low albumin sieving coefficient can conflict with each other. It is desirable to provide an outside-in fiber that achieves achieve both High KUF (or permeability) and essentially complete retention of albumin in the blood.

### SUMMARY OF THE INVENTION

In an embodiment of the invention, there may be provided a porous hollow fiber, the fiber comprising: a tubular body comprising a wall region and defining a lumen surrounded by the wall region, and the wall region comprising an outer surface, an inner surface, and a thickness extending in a radial direction from the outer surface to the inner surface, wherein the inner surface defines the lumen, the outer surface and the inner surface are generally concentric with each other, and the wall region and the lumen extend in an axial direction; wherein: the wall region comprises a porous fiber composition containing a mixture of a member of the polysulfone family, and polyvinylpyrrolidone; the wall region comprises a selective layer along the outer surface, and the selective layer is selective and allows the passage of small molecules such as urea and middle molecules e.g. β2 microglobulin, while excluding the passage of albumin therethrough so that the porous hollow fiber has a blood albumin retention coefficient of greater than 0.99 when measured in a direction from the outer surface to the inner surface; the wall region comprises a plurality of radially extending elongated macrovoids located in a portion of the wall region between the selective layer and the inner surface; and the porous hollow fiber has a permeability for water of at least approximately 6 mL/(h·mmHg·m²).

In an embodiment of the invention, there may be provided a porous hollow fiber, the fiber comprising: a tubular body comprising a wall region and defining a lumen surrounded by the wall region, the lumen having an inlet end and an outlet end, and the wall region comprises a mixture of a member of the polysulfone family, and polyvinylpyrrolidone, wherein: the wall region and the lumen extend in an axial direction from the inlet end to the outlet end; the wall region defines an outer surface, an inner surface, and a thickness extending in a radial direction from the outer surface to the inner surface, wherein the inner surface defines the lumen, and the outer surface and the inner surface are generally concentric with each other; the wall region comprises a first selective layer along the outer surface, wherein the first selective layer is selective for exclusion of passage of albumin therethrough so that the porous hollow fiber has an albumin sieving coefficient of less than approximately 0.05 when measured in a direction from the outer surface to the inner surface; the wall region comprises a second selective layer along the inner surface; the wall region comprises a plurality of generally radially extending elongated macrovoids extending between the first selective layer and the second selective layer, and wherein the porous hollow fiber has a permeability for water of at least approximately 6 mL/(h·mmHg·m²).

In an embodiment of the invention, there may be provided a porous hollow fiber, the fiber comprising: a tubular body comprising a wall region and defining a lumen surrounded by the wall region, and the wall region comprises a mixture of a member of the polysulfone family, and polyvinylpyrrolidone, wherein: the wall region and the lumen extend in an axial direction; the wall region defines an outer surface, an inner surface, and a thickness extending in a radial direction from the outer surface to the inner surface, wherein the inner surface defines the lumen, and the outer surface and the inner surface are generally concentric with each other; the wall region comprises a first selective layer along the outer surface, wherein the first selective layer has an average pore size of less than about 5 nanometers; the wall region comprises a second selective layer along the inner surface, wherein the second selective layer has an average pore size of less than about 10 nanometers; the wall region comprises a plurality of generally radially extending elongated macrovoids, wherein at least a portion of the plurality of radially extending elongated macrovoids extend to the inner surface, and the porous hollow fiber has a permeability for water of at least approximately 6 mL/(h·mmHg·m²).

In an embodiment of the invention, there may be a method of producing a hollow fiber, the method comprising: providing a bore liquid, a dope, a shower liquid and a coagulation bath; providing a triple concentric spinneret having a bore liquid channel and a dope channel annularly surrounding the bore liquid channel and a shower channel annularly surrounding the dope channel; causing the bore liquid and the dope and the shower liquid to flow through respective channels of the spinneret to form an emergent fiber; and stretching the emergent fiber as the emergent fiber passes through the coagulation bath while being pulled at a take-up velocity, wherein the triple concentric spinneret and the bore liquid and the dope and the shower liquid and the coagulation bath are all at respective temperatures that are substantially identical to each other or are within 2 to 10 degrees C. of each other, and wherein the shower liquid and the coagulation bath comprise respective higher concentrations of a non-solvent than does the bore liquid.

In an embodiment of the invention, there may be a method of producing a porous hollow fiber, the method comprising: forming an emergent fiber from a triple concentric spinneret having a bore liquid channel, a dope channel annularly surrounding the bore liquid channel, and a shower channel annularly surrounding the dope channel, by flowing a bore liquid through the bore liquid channel, flowing a dope liquid through the dope channel, and flowing a shower liquid through the shower channel; and stretching the emergent fiber as the emergent fiber passes through a coagulation bath while being pulled at a take-up velocity, wherein the triple concentric spinneret, the bore liquid, the dope, the shower liquid, and the coagulation bath are provided at temperatures within a 5 degrees C. range of each other, wherein the dope liquid comprises a member of the polysulfone family, a hydrophilic polymer, and a first organic solvent, the bore liquid comprises a second organic solvent and a first non-solvent, the shower liquid comprising a third organic solvent and a second non-solvent, and the coagulation liquid comprising a fourth organic solvent and a third non-solvent which may be water; wherein the first organic solvent, the second organic solvent, the third organic solvent, the fourth organic solvent are the same or different, wherein the first non-solvent, the second non-solvent, and the third non-solvent are the same or different, and wherein the shower liquid and the coagulation bath comprise respective higher concentrations of the non-solvent compared to the bore liquid.

In an embodiment of the invention, there may be provided a porous hollow fiber, the fiber comprising: a tubular body comprising a wall region and defining a lumen surrounded by the wall region, and the wall region comprising an outer surface, an inner surface, and a thickness extending in a radial direction from the outer surface to the inner surface, wherein the inner surface defines the lumen, the outer surface and the inner surface are generally concentric with each other, and the wall region and the lumen extend in an axial direction; wherein: an aspect ratio is defined as an outside diameter of the fiber divided by an inside diameter of the fiber, and the aspect ratio is less than 1.5; the wall region comprises a porous fiber composition containing a mixture of a member of the polysulfone family, and a hydrophilic polymer; the wall region comprises a selective layer along the outer surface, and the selective layer is selective for exclusion of passage of albumin therethrough so that the porous hollow fiber has a blood albumin retention coefficient of greater than approximately 0.95 when measured in a direction from the outer surface to the inner surface; the wall region comprises a plurality of radially extending elongated macrovoids located in a portion of the wall region between the selective layer and the inner surface; and the porous hollow fiber has a permeability for water of at least approximately 6 mL/(h·mmHg·m2).

### BRIEF DESCRIPTION OF THE ILLUSTRATIONS

Embodiments of the invention are further described but are in no way limited by the following illustrations.

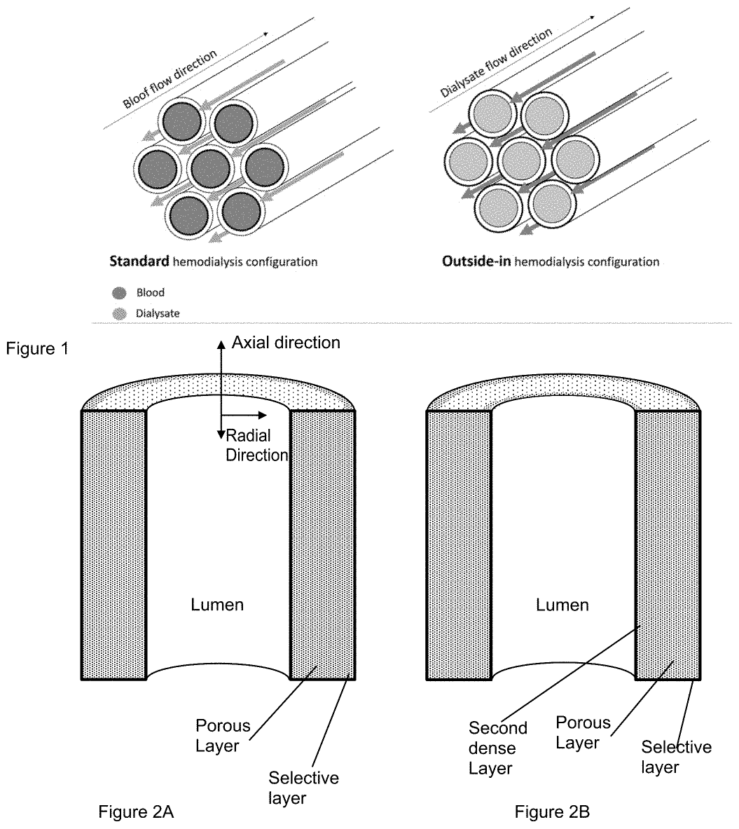

FIG. 1 is a schematic illustration comparing the conventional inside-out configuration with an outside-in configuration of an embodiment of the invention.

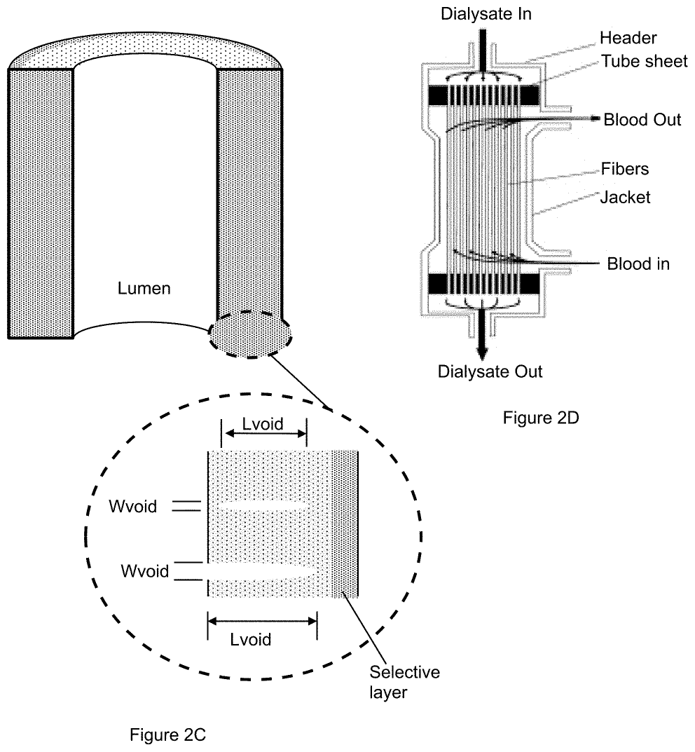

FIG. 2A is a schematic cross-sectional illustration of a hollow fiber of an embodiment of the invention, having a selective layer on the exterior. FIG. 2B is a schematic cross-sectional illustration of a hollow fiber of another embodiment of the invention, having a selective layer on the exterior and another selective layer on the luminal surface. FIG. 2C is a schematic cross-sectional illustration of a hollow fiber of an embodiment of the invention, having a selective layer on the exterior, similar to FIG. 2A, but additionally, there are shown two varieties of elongated macrovoids. FIG. 2C-1 illustrates an average pore size in the bulk layer of the hollow fiber. FIG. 2D shows, in cross-section, a dialysis cartridge comprising a plurality of the described hollow porous-walled fibers for use in the outside-in mode of operation.

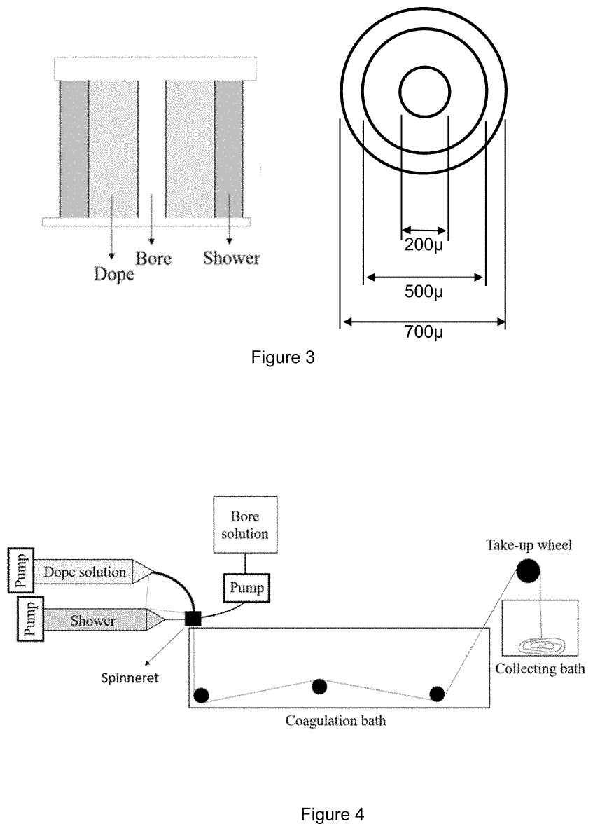

FIG. 3 shows a triple concentric spinneret used to manufacture the hollow fibers described herein.

FIG. 4 shows the overall arrangement of the spinneret, pumps, baths and take-up wheel for manufacturing the hollow fibers described herein.

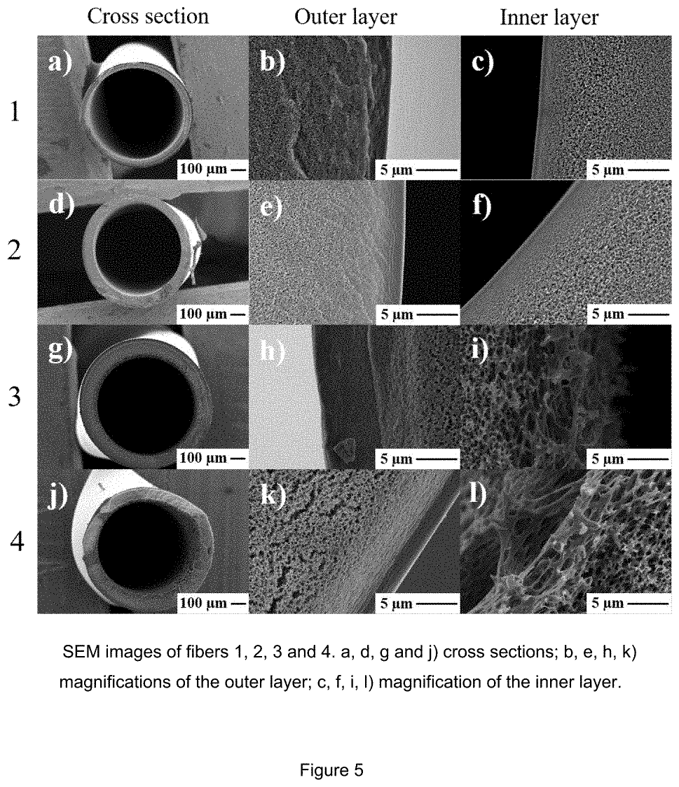

FIGS. 5-8 present Scanning Electron Microscope (SEM) images of the fibers that were produced, for all 16 of the experimental conditions that are reported herein.

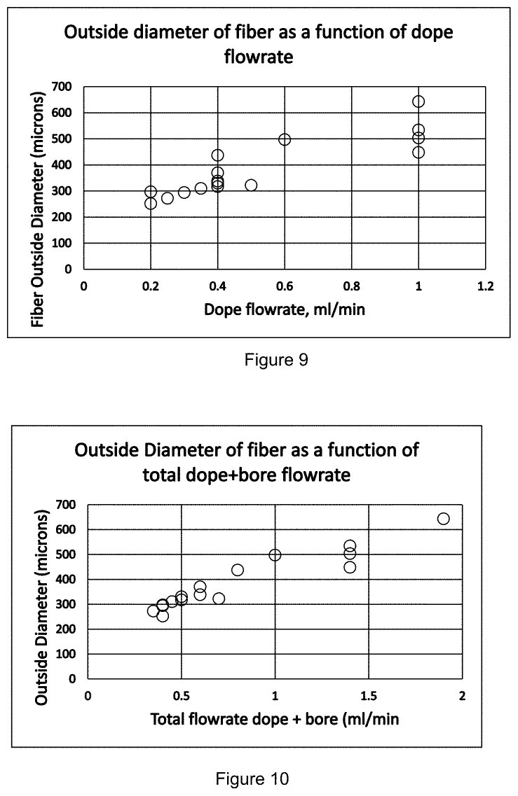

FIG. 9 is a plot, for all of the fibers produced, of the measured fiber outside diameter plotted as a function of the dope flowrate.

FIG. 10 is a plot, for all of the fibers produced, of the measured fiber outside diameter plotted as a function of the total flowrate of dope and bore liquid.

FIG. 11 is a plot, for all of the fibers produced, of the measured outside diameter as a function of a geometrically calculated outside diameter taking into account the total flowrate of dope and bore liquid, and also the speed of the take-up wheel.

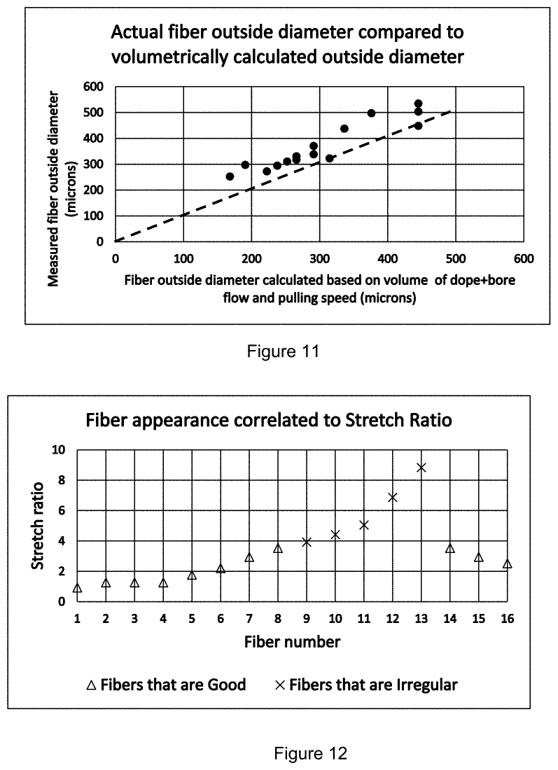

FIG. 12 shows a correlation between good or irregular appearance, as a function of Stretch Ratio.

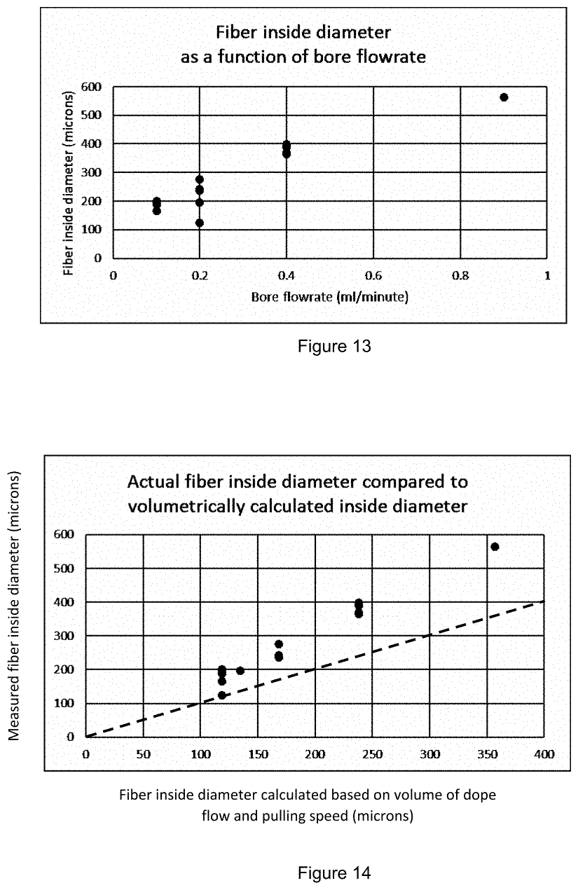

FIG. 13 shows a plot of measured fiber inside diameter as a function of bore flowrate only.

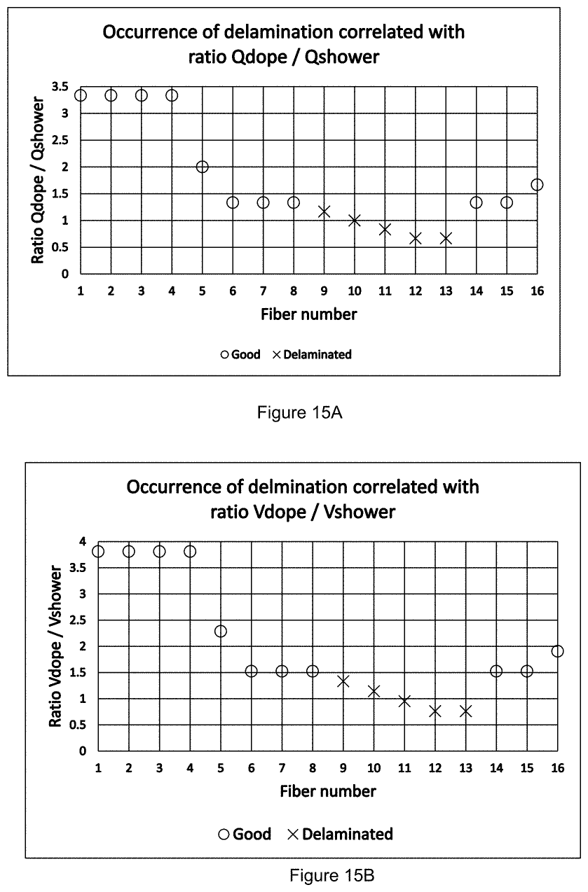

FIG. 14 is a plot, for all of the fibers produced, of the measured inside diameter as a function of a geometrically calculated inside diameter using the bore flowrate.

FIG. 15A illustrates a categorization of the fibers as irregular/delaminated or normal, as a function of the ratio of dope flowrate to shower flowrate. FIG. 15B illustrates a categorization of the fibers as irregular/delaminated or normal, correlated with the Speed Ratio.

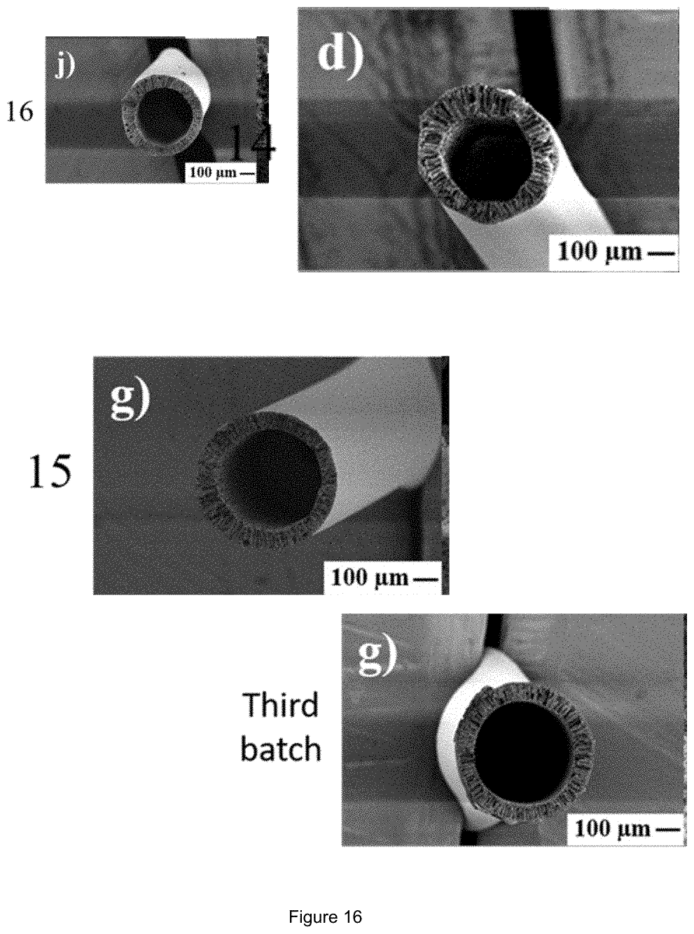

FIG. 16 illustrates various macrovoids.

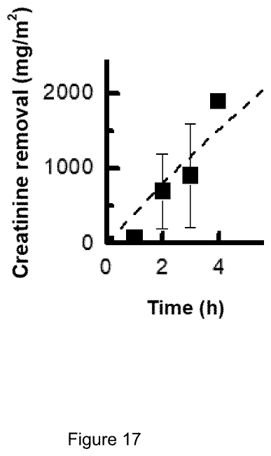

FIG. 17 illustrates experimental results for removal of creatinine.

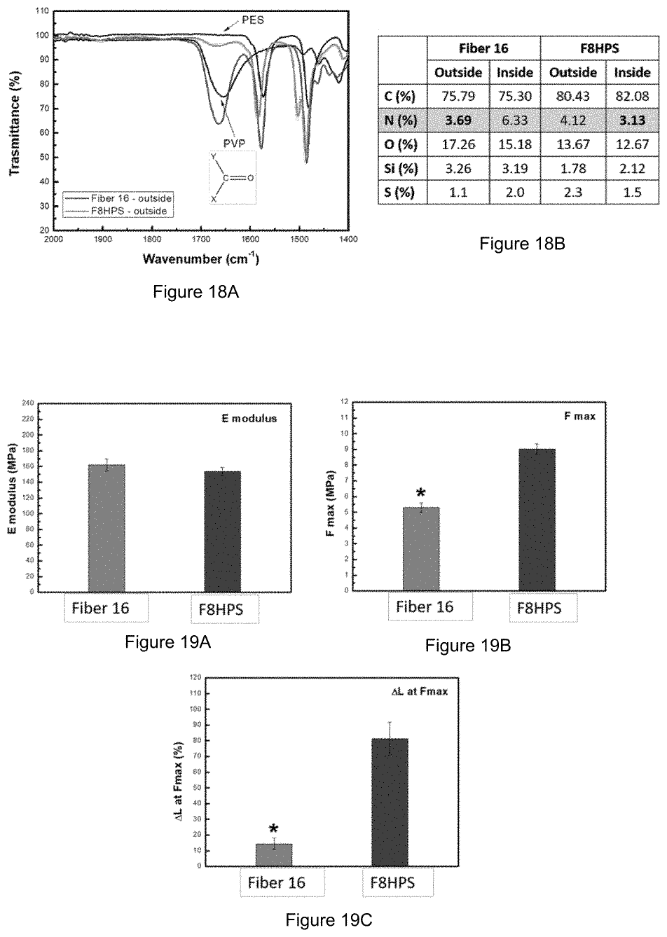

FIG. 18A shows ATR-FTIR results of fiber F16 and comparison to fiber F8HPS, for pure PES and pure PVP materials. FIG. 18B shows elemental molar percentage results of fiber F16 and fiber F8HPS measured by XPS.

FIGS. 19A-19C illustrate mechanical test results of an experimental fiber and also for a commercial fiber. FIG. 19A illustrates Young's Modulus; FIG. 19B illustrates Maximum strength before breakage; FIG. 19C illustrates Maximum elongation before breakage.

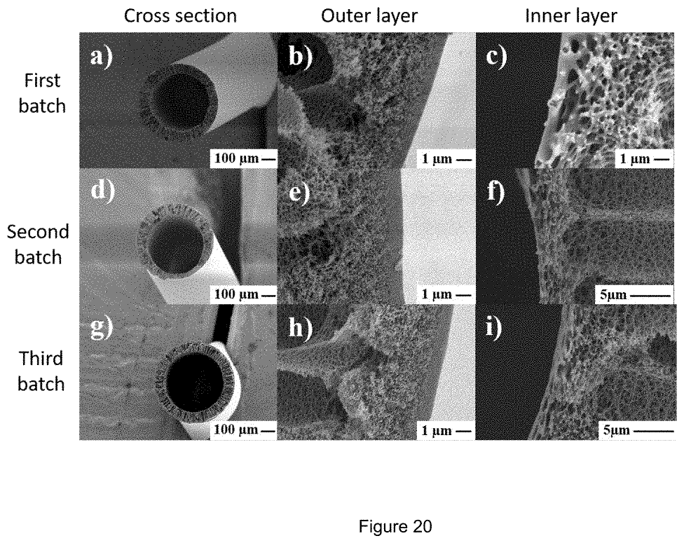

FIG. 20 shows Scanning Electron Microscope images of batches 1, 2 and 3 of fiber F15. Images a, d, g show cross-sections; images b, e, h show magnification of the outer layer; images c, f, i show magnification of the inner layer.

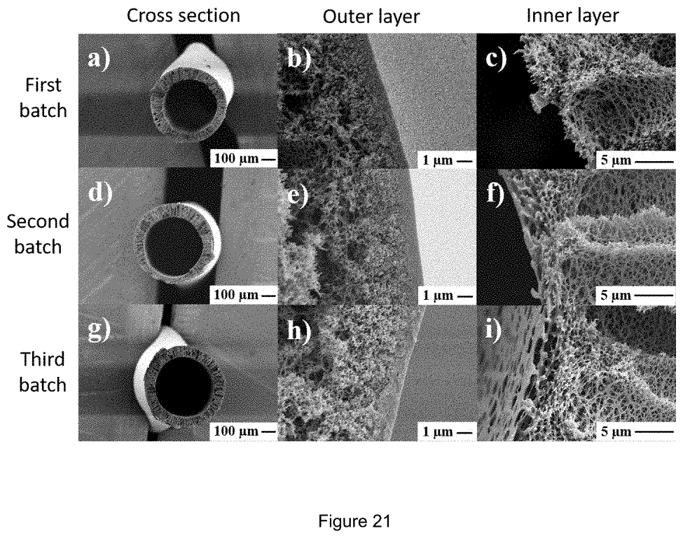

FIG. 21 shows Scanning Electron Microscope images of batches 1, 2 and 3 of fiber F16. Images a, d, g show cross-sections; images b, e, h show magnification of the outer layer; images c, f, i show magnification of the inner layer.

### DETAILED DESCRIPTION OF THE INVENTION

Effective hemodialysis treatment requires the removal of small molecular weight solutes such as urea, creatinine and salts, as well as middle molecules such as β2 microglobulin and protein bound solutes. In other words, it is desirable to achieve removal of both small molecules (by diffusive clearance) and middle molecules (by convective clearance) in order to provide effective treatment, which is usually accomplished by operating in the range of high flux dialyzers. At the same time, the hollow fiber dialysis membrane should have a molecular weight cutoff that substantially blocks the passage of larger essential molecules, especially albumin and other proteins. It also is desirable that overall the fiber wall or selective membrane layer have a sufficiently high permeability (such as for water). All of these performance parameters are influenced by the pore size and pore size distribution of the selective membrane layer, and are also influenced by the supporting layers or structure that constitutes the remaining thickness of the membrane wall other than the selective membrane layer. The term dialysis is intended herein to refer broadly to blood processing therapies including hemodialysis, and also hemodiafiltration, hemofiltration, slow continuous ultrafiltration, and other extracorporeal therapies.

U.S. Pat. Nos. 10,369,263 and 10,399,040 describe filter cartridges for dialysis and are assigned to Novaflux Inc., the assignee of the present application. The disclosures of U.S. Pat. Nos. 10,369,263 and 10,399,040 are incorporated herein in their entirety.

In mass transfer, the sieving coefficient is a measure of equilibration between the concentrations of two mass streams separated by a membrane. It is defined as the concentration of the mass receiving stream divided by concentration of the mass donating stream.

S=Cr/Cd

where
S is the sieving coefficient
Cr is the mean concentration in the mass receiving stream
Cd is the mean concentration in the mass donating stream

A sieving coefficient of unity implies that the concentrations of the receiving and donating stream equilibrate with each other. A sieving coefficient that is significantly smaller than unity represents a situation where the substance mostly does not pass through the membrane.

Uremic wastes or toxins are characterized by relatively low Molecular Weight, such as in the range of <1000 or <500 Daltons, which are considered small molecules. Examples of this are urea and creatinine. β2 microglobulin (having a Molecular Weight of around 11 kDa) is an example of a middle molecular weight substance that also needs to be removed from the blood during dialysis. It is intended that dialysis treatment should remove undesirable small and middle molecules from the blood.

It is desirable that albumin (which is a protein and has a molecular weight of about 67,000 Daltons) should be retained in the blood and should not be removed by the dialysis treatment. Thus, it is desirable that the albumin sieving coefficient be small, preferably as small as about 0.01. It is desired that the albumin not pass through or adhere to the membrane. There may also be other substances in a similar range of Molecular Weight that it are desirable to retain in the blood, similarly to albumin. These various criteria can be described in terms of a Molecular Weight Cut Off (MWCO). MWCO is an approximate boundary, in terms of Molecular Weight, between substances that either do or do not pass from the blood through the membrane into the receiving fluid.

In slightly greater detail, it may be understood that, in general there are two possible ways of losing substances, such as albumin, that are desired to remain in the blood. The first mechanism or mode of loss of such substances is for such substances to pass through the pores of the porous membrane, from the blood into the dialysate, which is what has just been described by the sieving coefficient. However, in addition to that mechanism, there is also another possible mechanism that could be active, namely adsorption of albumin or similar substances onto surfaces of the membrane. Substances that are adsorbed would not appear in the receiving fluid, and so the sieving coefficient measured as just described would not represent that material. Nevertheless, adsorbed substances still would be lost from the blood and unavailable to the patient. Both sieving and adsorption of substances such as albumin are undesirable. In order to adequately describe this situation, it is possible to define αs as the fraction of the substance of interest that passes through the pores (is sieved), αa as the fraction that is adsorbed, and αr as the fraction that remains in the blood. The relation among these is that αs+αa+αr=1. It is possible to define a new parameter which may be called the blood albumin retention coefficient, that describes the fraction of albumin that remains in the blood after loss of albumin resulting from either sieving or adsorption. If there is no adsorption of albumin inside the wall of the fiber, then the blood albumin retention coefficient αr is simply 1-albumin sieving coefficient (the complement of the albumin sieving coefficient). However, more generally, the blood albumin retention coefficient αr is αr=1−αs−αa Sieving is minimized by strategies such as providing small pores. Adsorption is believed to be minimized by strategies such as providing a smoother surface that is hydrophilic and hemocompatible and by providing a wall thickness that is not larger than necessary. In experimentation described herein, the parameter that is measured experimentally is the albumin sieving coefficient.

Permeability describes the flowrate of liquid through a membrane per unit of membrane area and per unit of pressure drop driving the flow. KUF is, for a particular dialyzer, the flowrate of liquid through the membrane per unit of pressure drop driving the flow. Dialyzers are usually categorized as either High Flux or Low Flux. Currently the majority of hemodialysis is performed using high flux dialyzers. The US Food and Drug Administration considers high-flux dialyzers to be dialyzers that have a KUF of at least 12 mL/(h·mmHg). (This describes the performance of a particular dialyzer rather than the membrane itself.) A newer proposed definition is reported to be that a high flux dialyzer has a KUF of >14 mL/(h·mmHg) in conjunction with a certain requirement relating to clearance of β2 microglobulin. A typical dialyzer for adult human hemodialysis has a surface area in the range of 1.5 m² to 2.0 m², so on a basis of unit area of the membrane, this corresponds to a permeability of 6 to 8 mL/(h·mmHg·m²). The European Dialysis (EUDIAL) working group defines high-flux dialyzers as having a permeability of >20 mL/(h·mmHg·m²). (Ref.: Claudio Ronco and William R. Clark, Haemodialysis membranes Nature Reviews|Nephrology, June, 2018, vol. 14, pp. 394-410)

A conventional high-flux dialyzer is often used in a situation of having a bidirectional convective flow through the fiber wall. The bidirectional convective flow arises from the transmembrane pressure applied across the fiber dialyzer during hemodialysis. At some portion along the length of the fiber, the transmembrane pressure (TMP), i.e., pressure drop across the fiber, is such as to convectively drive liquid outward from the blood through the fiber wall into the dialysate. At some other portion along the length of the fiber, the direction of the TMP is reversed and is such as to convectively drive liquid inward from the dialysate through the fiber wall into the blood (referred to as backfiltration). This process is also called internal filtration as is known in hemodialysis. The composition of aqueous liquid that is transported outward through the fiber wall due to transmembrane pressure (convection) is different from the composition of the aqueous liquid that is transported inward through the fiber wall due to the transmembrane pressure difference in the opposite direction. Flow of liquid through the membrane under transmembrane pressure is the driving force for convective transport, which is helpful for the clearance of middle molecules during dialysis by what is called convective clearance. During the mass transfer in both of those directions in which convective transport takes place, diffusive transport is always in the direction from a higher concentration to a lower concentration because it depends on concentration gradient only.

In contrast, in a low flux dialyzer, the convective transport is limited and, because of the small pore size, the convective clearance of middle molecules is almost non-existent. In a low flux dialyzer, the mass transport is almost entirely transport of small molecules by diffusion. The preferred dialysis therapy now is to use high flux dialyzers, which simultaneously provides effective diffusive and convective clearance for uremic toxins.

In general, there is some tradeoff among the goals of having high permeability and retaining certain sizes of molecules in the blood. Achieving good blood retention of molecules such as albumin is associated with providing a smoother selective layer that has small pores. Further details are related to the thickness of the selective layer and the structure of the remainder (non-selective) portion of the wall of the hollow fiber. It is furthermore important that the selective layer be essentially intact and physically robust, so that there are no cracks or holes through the selective layer. At the same time, small pores tend have high flow resistance or low permeability to the passage of liquids in general through them. The permeability is also influenced by parameters such as the thickness of the selective layer, and by the structure of the non-selective portion of the fiber wall that serves as a supporting structure for the selective layer. In general, the small pores that provide the Molecular Weight selective properties of the selective layer also create flow resistance, making it more difficult to achieve a high flux or high permeability membrane. In general, in order to achieve high permeability for flow of liquid through the fiber wall, it is desirable that the selective layer be as thin as possible, and it is desirable that the remainder of the fiber wall (supporting structure) be more open and have high permeability. The properties of the selective layer and of the supporting layer, such as thickness, pore size, and other parameters, are determined by the material of construction and by various parameters of the manufacturing process acting in combination. Hemocompatibility of the external surface is determined by a combination of small pore size and small surface roughness on the exterior surface and the presence of a hydrophilic polymeric chemical constituent such as PVP, which is discussed elsewhere herein.

In current clinical practice, the fiber is used in an inside-out configuration, in which blood flows inside the lumen of the fiber while dialysate flows on the outside of the fiber. Fibers that are designed for this conventional inside-out configuration usually have a selective membrane layer, which is a smaller-pore, more-dense layer, that is on the interior or luminal surface, which is the blood-facing surface. Such a surface characteristic advantageously is made smooth and hydrophilic to discourage activating the complement system or promoting thrombosis which would lead to the formation of blood clots inside the fiber lumen.

In essentially all current hemodialysis practice, the hollow fiber membrane is operated in the inside out configuration and the selective membrane layer is located at the luminal surface of the fiber.

In contrast to conventional fibers, embodiments of the invention are useful for operating in an outside-in configuration. For outside-in filtration, it is desirable or even necessary for the outside surface of the fiber to have the selective layer and to have properties of hemocompatibility, which are hydrophilicity and small surface roughness.

In regard to the outside-in configuration, one of the few examples of a fiber that has been developed for outside-in filtration is Krause and Gohl's patent EP 2083939 B1, and similarly U.S. Pat. No. 8,596,467 to Krause and Gohl et al. As shown in the images of electron microscopy in Krause and Gohl's patent, the smallest pores exist at the outer selective layer for all the illustrated example fibers shown in Krause and Gohl. In regard to the sieving of albumin, in paragraph 0013 of Krause and Gohl's Patent, it is stated that Krause and Gohl's fiber exhibits a “high diffusive transport in a broad range with respect to the molecular size (up to 100000 Dalton).” Thus, albumin, which has a Molecular Weight of about 67,000 Daltons, would definitely be able to pass through the wall of such a fiber, which is undesirable.

In regard to permeability, the range of permeability of Krause and Gohl's fiber is described in U.S. Pat. No. 8,596,467 as “In a further embodiment the hollow fiber membrane has a hydraulic permeability within the range of 1×10⁻⁴-100×10⁻⁴[cm3/cm2×bar×s], preferably within the range of 1×10⁻⁴ to 70×10⁻⁴ [cm³/cm²*bar*s], and most preferably within the range of 1×10⁻⁴ to 27×10⁻⁴ [cm³/cm²*bar*s]. With this hydraulic permeability the convective transport through the membrane wall is minimized at the same time having high diffusive transport in a broad range . . . ” Expressed in other units, these permeability values are: [4.7 to 474 mL/(m²*mmHg*h)]; preferably [4.7 to 331 mL/(m²*mmHg*h)]; most preferably [4.7 to 331 mL/(m²*mmHg*h)]. This quantitative description indicates that Krause and Gohl's permeability is such that the fiber could be just slightly below the range of a High Flux dialyzer, or could be well within the range of a High Flux dialyzer. Nevertheless, especially because of its albumin leakage, the fiber of Krause and Gohl does not meet the goals of embodiments of the present invention.

The outside-in fiber of Krause and Gohl is illustrated in FIG. 2B of Krause and Gohl for a typical one of Krause and Gohl's fibers. The fiber is described as comprising five successive layers with each layer having a different density, with the outermost layer being the most dense layer. On its outside surface, Krause and Gohl's fiber has a relatively thin layer of more-dense porous material, and in the remainder of the wall region it has a less-dense porous material. The thickness of the more-dense layer can be estimated from the visual appearance of Scanning Electron Microscope photographs of cross-sections of the fibers, together with the dimensional scale bar in those photographs. It can be estimated from the various photographs in Krause and Gohl that the thickness of his dense layer is approximately 2 to 4 microns. It can also be noted that Krause and Gohl's fiber does not contain the elongated macrovoids that are found in many high-flux fibers.

Another patent about an outside-in fiber is Gorsuch 20070023353 (issued as U.S. Pat. No. 7,585,412), which is directed at applications to plasmapheresis (plasma separation) and ultrafiltration. The disclosed fiber has a selective layer on outside, similar to what is disclosed in Krause and Gohl. Albumin retention and MWCO are not specifically described, but the patent describes that for ultrafiltration (which uses a more selective membrane than plasmapheresis), the pore size in the dense layer could be as small as 3-6 nanometers, which might be appropriate to hold back albumin. The permeability data indicates performance in the high flux range. There is no disclosure of elongated macrovoids.

In still other applications, outside-in fibers are known for purposes of water filtration, but in addition to they usually are not selective enough to hold back albumin.

It can be understood that, depending on whether a fiber is intended for a commercial water filtration application or for a dialysis application, there are differences in the ranges of certain design parameters. The fibers intended for water filtration typically operate in an environment in which the outside-in flow through the fiber walls is driven by a significantly large external pressure, for example, many atmospheres. In contrast, in a blood processing application, such large pressures are not acceptable. In blood processing situations such as dialysis, practical considerations dictate that fibers for dialysis experience only a small external pressure (substantially less than one atmosphere) (if they experience any external pressure at all).

For a fiber that is subject to external pressure, there are considerations of compressive stress in the wall and also consideration of elastic stability appropriate to resist buckling due to the external pressure. It is believed that in situations of present interest, elastic stability is the more governing consideration. The elastic stability limit of an externally pressurized tube is influenced quite strongly by the ratio of wall thickness to fiber radius. A formula for the elastic stability limit in a simple situation is p=0.25*(E/(1−ν²))*(t/r)³, where E is Young's Modulus, ν is Poisson's Ratio, t is thickness of the wall, and r is average radius of the wall, and p is the external pressure corresponding to the elastic stability limit. The average radius of the wall is often considered to be the average of the inside radius and the outside radius. This formula is applicable to thin-walled tubes having no constraint at their ends. According to this formula, other than material properties such as Young's modulus and Poisson's Ratio, the only parameter influencing the elastic stability limit is the ratio t/r, and the dependence is to the third power. The parameter t/r is essentially related to the aspect ratio (the ratio of outside diameter to inside diameter).

As described by this formula, to the extent that the wall thickness is determined by considerations of external pressurization elastic stability, water filtration fibers are thicker-walled than dialysis fibers. For dialysis fibers, given that the wall does not need to be very thick for withstanding external pressure, there is incentive to reduce the wall thickness in order to provide greater permeability for liquids passing through the wall. Therefore, the dialysis fiber wall thickness tends to be notably thinner than is the case for water filtration fibers. For dialysis fibers, a typical outside diameter is 250 microns, and a typical wall thickness is 20 to 40 microns (which gives an inside diameter of 170 to 210 microns). These combinations of dimensions result in an aspect ratio of 1.19 to 1.47.

There are also certain overall fiber dimensions such as outside diameter which differ between water filtration fibers and dialysis fibers. In absolute dimensions, water filtration fibers tend to have larger outside diameters than fibers for dialysis. In the case of dialysis fibers, the smaller outside diameter of dialysis fibers is favorable for achieving a large total fiber surface area in a reasonable set of overall dimensions of a dialyzer cartridge. Fibers for dialysis generally do not have outside diameters greater than about 300 microns.

It is further believed that typical fibers for water filtration are less selective than the fibers of embodiments of the invention. i.e., they would allow passage of substantial amounts of albumin.

Another example of a known dialysis fiber is the conventional fiber described in Buck and Goehl U.S. Pat. No. 8,136,675 and WO2004056460, which is a conventional inside-out fiber. The patents refer to “the dialysate surrounding the hollow fibres during use,” and they state that “In the innermost layer of the hollow fibre a separation layer is present, having a thickness of <0.5 μm and containing pore channels, having a pore size of 15-60 nm, preferably 20-40 nm.” This fiber has a selective layer on the luminal side and, located radially outward from that selective layer are elongated macrovoids. The selective membrane layer is disclosed as having a thickness of less than 0.5 microns. This fiber allows passage of molecules up to 45000 Daltons and has an exclusion limit of about 200,000 Daltons, and it is described as having a sieving coefficient for albumin in presence of whole blood that is below 0.05. The hydraulic permeability for various fibers of the invention is given as 218 or 190 or 54*104 cm/s/bar (which converts to 1032 or 900 or 256 mL/m²*mmHg*h, which would be considered high flux). Again, this is an inside-out fiber (which is the conventional orientation), having its main selective layer at the luminal surface.

For porous-walled hollow fibers used in dialysis, it is common to use members of the polysulfone family. It should be appreciated that the polysulfone family can be more conveniently referred to as a polysulfone polymer. The polysulfone family includes polysulfone, polyethersulfone, and polyarylethersulfone, and derivatives thereof which can be more conveniently referred to as polysulfone derivatives. Often such material is combined with another more hydrophilic material such as polyvinylpyrrolidone. Another possible hydrophilic material or additive is polyethylene glycol (PEG). One of the commonly used combinations of materials is a combination of polyethersulfone (PES) and polyvinylpyrrolidone (PVP). The polyethersulfone serves as a base polymer responsible for the overall structure and the desired mechanical properties, and the polyvinylpyrrolidone additive serves as a hydrophilic agent and has a role in the formation of the porosity and microstructure of the supporting porous layer. These two polymeric substances both are soluble in organic solvents such as n-methyl pyrrolidone (NMP) or similar organic solvents. Typically, a composition containing the two polymers in a solvent is a viscous or viscoelastic liquid suitable for extruding or spinning through a spinneret. This liquid is referred to as “dope.” It can be understood that, alternatively, other polymers or other polymer families or other combinations of polymers, or other solvents, could be used. Polysulfone derivatives include those polysulfone polymers that are modified to increase or enhance hydrophilic properties. Exemplary modifications include surface modification and chemical modification by adding, for example, chemical groups to the polymer. Exemplary derivatives and techniques are described, for example, in Alenazi et al., Modified polyether-sulfone membrane: a mini review, Designed Monomers and Polymers, 20:1, 532-546, the entire disclosure of which is incorporated herein by reference.

The hydrophilic polymer may include any of polyvinylpyrrolidone (PVP), copolymers of polyvinylpyrrolidone, polyethylene glycol, polypropylene glycol, polyethylene oxide, other hydrophilic polymers, and mixtures thereof. Other polymer having hydrophilic properties, in addition to these named types of polymers, can be used as the hydrophilic polymer in combination with the polysulfone family or polysulfone polymer.

In regard to manufacturing method, it is common that during extrusion or spinning, a surface of the dope is exposed to another substance that contains either organic solvent or water or a mixture of both. Typically, to form the dope, the polymer(s) are dissolved an organic solvent such as n-methyl pyrrolidone (NMP) or similar organic solvents. There are other substances referred to as non-solvent, in which the polymer or a significant component thereof does not have large solubility. A most common non-solvent may be water, but other examples include isopropanol, glycerol, and mixtures of any of these non-solvent substances. The solvent and the non-solvent may be miscible with each other. The surface of the emerging fiber, or particular surfaces of the emerging fiber, can be exposed to various solutions of solvents and non-solvents as desired, or to air, to influence the phase separation process and the morphology of the resulting fiber. Exposure can occur in the lumen, on the exterior of the emerging fiber, in the air gap, and in the coagulation bath.

It is known that, depending on the process details, there can be formed various combinations of a dense layer having small pores, a spongy region having pores that are larger than those in the dense layer, and elongated macrovoids, which are elongated and still larger than any other pores. It is known that in general, exposing a surface of the extruded dope to a composition that is rich in organic solvent delays the phase separation speed between the spinning dope and the coagulating liquid and thus promotes the formation of open or larger pores, resulting in larger internal pore size near the surface of the hollow fiber membrane that is exposed to that substance. In contrast, exposure to a composition that has a high concentration of water (or more generally a composition having a high concentration of a non-solvent) and little or no organic solvent promotes rapid solidification and the formation of smaller more densely populated pores.

In regard to the formation of elongated macrovoids, it is believed that the formation of such macrovoids is influenced by variables such as the concentration of solvent and non-solvent of the liquid contacting a surface of the emerging fiber, and the speed of the spinning process, and temperatures. The formation of elongated macrovoids during membrane production involves several different mechanisms occurring simultaneously. One of the mechanisms/parameters affecting macrovoid formation is the speed of phase separation. Membranes that experience (when the solvent/non solvent exchange is fast) immediate liquid-liquid demixing tend to exhibit macrovoids, whereas membranes that experience delayed demixing tend to exhibit spongy-like structures (usually when a mixture of solvent and non-solvent is in contact with a particular surface). Thus, elongated macrovoids are generally the consequence of a fast phase separation. In regard to other parameters, usually, macrovoids gradually appear with an increase of the membrane wall thickness. If the fiber exterior in the air gap is exposed to atmosphere, moisture in the air gap region can promote the formation of macrovoids. If the viscosity of the dope solution is increased, that increase in the viscosity of the dope solution can decrease the formation of macrovoids. Also, the amount and the molecular weight of the polyvinylpyrrolidone present in the dope can influence this.

### Embodiments of the Invention

An embodiment of the invention can be a fiber that can be used in hemodialysis in an outside-in configuration, in which blood flows on the outside of the hollow fiber and dialysate flows on the inside of the hollow fiber. In FIG. 1, a dialysis cartridge operated to perform outside-in filtration is illustrated and contrasted with a conventional dialysis cartridge operated to perform inside-out filtration. In embodiments of the invention, the exterior surface of the fiber is blood-facing. This configuration offers advantages in regard to the ability of blood to find alternate flowpaths in the inter-fiber space if a clot should form, and this offers the prospect of greatly increased operating time for an individual dialysis cartridge.

In an embodiment of the invention, a porous-walled hollow fiber may comprise a tubular body comprising a wall region and defining a lumen surrounded by the wall region. The lumen may have an inlet end and an outlet end, and the wall region and the lumen may extend in an axial direction from the inlet end to the outlet end. The wall region may define an outer surface, an inner surface, and a wall thickness extending in a radial direction from the outer surface to the inner surface. The inner surface may define the lumen. The tubular body may be of generally circular cross-sectional shape, and the outer surface and the inner surface may be generally concentric with each other. The wall region may comprise a first selective layer, or outer selective layer, along the outer surface, wherein the first selective layer is selective for exclusion of passage of albumin therethrough so that the porous hollow fiber may have a blood albumin retention coefficient described herein, when measured in a direction from the outer surface to the inner surface. In some embodiments, the wall region may comprise a second selective layer, or inner selective layer, along the inner surface. The portion of the wall region that is not the first or outer selective layer or the second or inner selective layer can be referred to as the bulk layer. In addition, there is no requirement that the wall region includes an inner selective layer. In fact, the wall region may include a film or layer along the inner surface that does not possess significant selective or screening properties for exclusion of passage of albumin therethrough.

Referring now to FIG. 2A, in an embodiment of the invention, a porous-walled hollow fiber can have a dense small-pore porous selective layer (a first selective layer) that is on the exterior surface of the hollow fiber. In an embodiment of the invention, the inner (lumen) surface of the hollow fiber may possess higher porosity and be free of a dense porous selective layer. In an embodiment of the invention the selective layer, which is the outer layer, may be a dense porous layer having a thickness of less than approximately 1 micron (μm). In an embodiment, the outer surface of the hollow fiber may be hemocompatible, which means that it has the properties of being made from safe polymers that do not activate thrombosis or induce complement activation, being hydrophilic, soft, hydrated and having a surface roughness of the blood-facing surface being smaller than 10 nanometers or smaller than 20 nanometers root-mean-square. Roughness can be measured by an atomic force microscope for example. Hydrophilic, for this purpose, may be considered to mean having a surface contact angle with pure water that is less than 60 degrees, or less than 50 degrees, or less than 40 degrees.

Referring now to FIG. 2B, there is illustrated a porous-walled hollow fiber similar to what is illustrated in FIG. 2A, except that there is additionally a second dense layer on the luminal surface of the fiber. The second dense layer could in general be different from the selective layer illustrated in FIG. 2A. It could differ in parameters such as pore size, pore size distribution, thickness of the layer, or any other parameters as may be desired. Alternatively, if desired, the second layer could be the same as the layer on the external surface. For example, the wall region may comprise, along the outer surface, a first selective layer having an average pore size of less than about 5 nanometers, and may comprise, along the inner (luminal) surface, a second selective layer having an average pore size of less than about 10 nanometers. It is believed that the second dense layer could be useful as a secondary defense against entry of endotoxins into the patient's blood such as during the backfiltration portion of the dialysis process. It is pointed out that the porous layer, that does not include the selective layer, can be referred to as the bulk layer.

It can further be noted that having a dense layer on the luminal surface also, as illustrated in FIG. 2B, may be useful for ensuring the cleanliness of backfiltration fluid that enters the patient's blood. Still further, it is even possible that, using such a fiber, it might be possible to make a single dialyzer design that could be used in either inside-out filtration or outside-in filtration.

Reference is now made to FIG. 2C, which is similar to FIG. 2A except that it additionally illustrates elongated macrovoids within the porous region of the wall. A macrovoid may be considered to be a region that is substantially empty space, which is larger than the pores that make up other portions of the wall. For example, a macrovoid may have a dimension that is at least five times as large, or ten times as large, as the average dimension of pores that are adjacent to it. Macropores may be elongated in one direction compared to other directions. The elongated macrovoids may extend generally in a radial direction, which is the direction from the inner surface of the wall to the outer surface of the wall. One such elongated macrovoid is illustrated as being entirely contained within the wall and not touching the selective layer. It is illustrated in FIG. 2C that a porous or spongy region exists between the elongated macrovoid and the selective layer. A porous or spongy region also exists between the elongated macrovoid and the luminal surface of the fiber. Also illustrated in FIG. 2C is an elongated macrovoid that breaks through to the luminal surface of the fiber so that the elongated macrovoid is in communication with the luminal space of the fiber. For both illustrated types of elongated macrovoids, the elongated macrovoid does not touch the selective layer; rather it is separated from the selective layer by a spongy region. Either type of such elongated macrovoids may have a shape defined by a radially extending dimension and a transverse dimension that is perpendicular to the radial dimension. For an elongated macrovoid that does not intersect the luminal wall, the radially extending dimension may extend from one end of the elongated macrovoid to the other end of the elongated macrovoid. For an elongated macrovoid that does intersect the luminal wall, the radially extending dimension may extend from one end of the elongated macrovoid to the luminal surface. The transverse dimension of the elongated macrovoid may be a maximum transverse dimension found among transverse dimensions at various places along the radial dimension. The transverse dimension of the elongated macrovoid may be a transverse dimension measured at a midpoint along the radial dimension. The macrovoids are illustrated in contrast to pores in the porous layer or bulk layer in FIG. 2C-1 where the average pore size in the porous layer or bulk layer is identified. This average pore size is based on an average of pores that does not include the macrovoids. Also, FIG. 2C illustrates an exemplary macrovoid that is separated from both the lumen and the selective layer by at least a portion of the bulk layer, and also illustrates an exemplary macrovoid that is open to the lumen but is separated from the selective layer by at least a portion of the bulk layer. It should be appreciated that the bulk layer helps support the selective layer, and if the fiber includes an inner selective layer, the bulk layer can be provided to support the inner selective layer.

In an embodiment of the invention, the elongated macrovoids may have a radial dimension that is about 30% to 90% of the wall thickness of the fiber wall. For example, for a typical wall thickness of the fiber wall that might be 40 microns, the radially extending dimension of elongated macrovoids might range from 12 microns to about 36 microns. In embodiments of the invention, the radially extending dimension of an elongated macrovoid may be at least two times, or at least three times, or at least five times the transverse dimension. In an embodiment of the invention, the wall region may comprise a density of the elongated macrovoids, as evidenced by microphotographs in the Examples elsewhere herein, such that proceeding around the circumference of the fiber, there are approximately 50 to 100 elongated macrovoids spaced around the entire circumference. Given the fiber diameters (such as an outside diameter in the range of 300 microns) and other dimensions described in the Examples herein, this corresponds to an approximate macrovoid spacing of about 5 to 10 microns from the centerline of one elongated macrovoid to the centerline of a neighboring elongated macrovoid. Allowing for some wall thickness, the transverse dimension of an elongated macrovoid may be in the range of from 3 to 8 microns. This macrovoid-to-macrovoid spacing just discussed and estimated is a spacing in the circumferential direction. With regard to the axial direction, it is believed that the elongated macrovoids form similar repeated units similarly spaced along the axial direction. However, this is not definitely known, and it is possible that the nature of structure repetition along the axial direction might not be exactly the same as the nature of structure repetition along the circumferential direction.

In an embodiment of the invention, the hollow fiber may comprise a member of the polysulfone family. The family includes polysulfone, polyethersulfone (PES) and polyarylethersulfone. The polymer system may also include a hydrophilic polymer such as polyvinylpyrrolidone (PVP), which may be of any desired molecular weight in any desired polydispersity or molecular weight distribution, and the molecular weight distribution could be either unimodal or bimodal. In this polymer system, the polyethersulfone, which may be present in a larger proportion, may provide structure, and the polyvinyl pyrrolidone (PVP) may serve to make the polymer combination more hydrophilic, in particular at the blood-facing surface (outer surface) of the hollow fiber. The PVP may also influence the process of phase separation of the polymer from the solvent. Another substance that can serve the same purpose as PVP is polyethylene glycol (PEG). Other hydrophilic polymers are also possible.

In hollow fibers for hemodialysis applications, typical dimensions are an outside diameter in the range of 200 to 300 microns and a fiber wall thickness which may be 20 to 40 microns. A typical ratio of the outside diameter to the inside diameter may be about 1.25. This means that the surface area of the outside surface of the hollow fiber is about 1.25 times the surface area of the lumen of the hollow fiber. This means that if the selective layer is on the outside surface of the fiber, the surface area that is involved in dialysis, sieving and filtration is about 1.25 times the surface area of what would be the selective layer area if the selective layer were on the lumen surface of the fiber. This feature provides correspondingly more filtration performance for the same fiber dimensions and quantity of fibers and cartridge dimensions, compared to what is available for an inside-out configuration. In an embodiment, placing the selective membrane layer on the exterior of the fiber may increase the total surface area of the membrane, which would provide more effective dialysis therapy when that same number and dimensions of fibers is used to make the dialyzer.

An embodiment of the invention may further include a dialysis cartridge comprising a plurality of the described hollow porous-walled fibers, and further comprising: a housing having a housing interior including a housing midsection interior region, a housing blood supply port, a housing blood discharge port; a first end barrier that joins with the fibers at first ends of the fibers and joins with the housing interior of the housing and bounds a first end plenum and separates the first end plenum from the housing midsection interior region; a second end barrier that joins with the fibers at second ends of the fibers and joins with the housing interior and bounds a second end plenum and separates the second end plenum from the housing midsection interior region, wherein a blood flow compartment comprises an inter fiber space defined by the fiber exteriors and an interior housing surface along the housing midsection interior region, the housing blood supply port and the housing blood discharge port, the inter fiber space, the housing supply port, and the housing discharge port being in fluid communication with each other, and wherein a fluid flow compartment comprises the first end plenum, the fiber interiors, and the second end plenum, the first end plenum, the fiber interiors, and the second end plenum being in fluid communication with each other. Such a cartridge is schematically illustrated in FIG. 2D.

In an embodiment of the invention, a porous-walled hollow fiber with a selective membrane layer located on the outside surface of fiber can have a blood albumin retention coefficient of 0.97 or greater (corresponding to an albumin sieving coefficient less than 0.03 if there is no adsorption) and preferably a blood albumin retention coefficient of 0.98 or greater (corresponding to an albumin sieving coefficient less than 0.02 if there is no adsorption), and more preferably a blood albumin retention coefficient of 0.99 or greater less (corresponding to an albumin sieving coefficient less than 0.01 if there is no adsorption). Even more preferably, the fiber could have a blood albumin retention coefficient of 0.997 or greater (corresponding to an albumin sieving coefficient less than 0.003 if there is no adsorption) or could have a blood albumin retention coefficient of 0.999 or greater (corresponding to an albumin sieving coefficient less than 0.001 if there is no adsorption). In an embodiment of the invention, a porous-walled hollow fiber can have a permeability greater than 6 mL/hr mmHg m², or greater than 20 mL/hr mmHg m². In an embodiment of the invention, a fiber can have a supporting structure that comprises elongated macrovoids that open toward the lumen-side of fiber while not interrupting the outer (outside) selective layer. In an embodiment, the supporting porous layer along with the selective membrane layer may provide a fiber with mechanical strength of at least approximately 5 MPa at breakage, or an elongation strain of at least approximately 10% at breakage, or a Young's Modulus of approximately 160 MPa.

A spinneret used in experiments herein is illustrated in FIG. 3. A spinneret in general comprises a central bore or nozzle that is surrounded by a first annular region. In spinnerets such as the illustrated spinneret, additionally the first annular region in turn may further be surrounded by yet another annular region which is a second annular region. During extrusion or spinning, the first annular region may be what eventually forms the wall of the hollow fiber, which may be formed from the dope. Within the bore there may be, co-extruded with the dope, a bore liquid that is in contact with the inner surface of the dope and occupies and defines the bore space to help maintain the size and shape of the lumen, and also may influence physicochemical processes that occur during the spinning process. The bore material may interact with the dope thermally or chemically or both and may or may not influence phase separation processes. Outside the first annular region, in contact with the outer surface of the dope of the emerging wall of the hollow fiber, there may be a “shower” extruded from the second annular region. The “shower” may surround the emerging fiber until all of the extruded material reaches the coagulation bath. The shower may interact with the dope thermally or chemically or both, and, importantly, may influence phase separation processes so as to form the desired microstructure and porosity.

Various processing parameters during the spinning process may influence the morphology and dimensions of the manufactured fiber. In manufacturing situations that involve temperature differences, the extruded or spun material may emerge from the spinneret exit at an elevated temperature and may cool or solidify as time progresses and as the material moves onward. This may give rise to the use of the term “quench.” Also, the fiber may pass through a coagulation bath of liquid between the spinneret and the take-up wheel. The coagulation bath may interact with the dope thermally or chemically or both and may influence phase separation processes so as to form the desired microstructure and porosity. Still other adjustable parameters are the dope flowrate and the bore flowrate and the shower flowrate. These parameters and their interrelationships and ratios are discussed elsewhere herein.

Typically there is some distance between the spinneret exit and the liquid in the coagulation bath, and this space is often referred to as an air gap. If the spinneret only comprises a bore and a dope region, then the outside surface of the extruded spun fiber is likely to be exposed to air as it traverses the distance between the spinneret exit and the coagulation bath, i.e., the air gap. As described herein, in an embodiment of the invention, the spinneret may comprise a second annular region surrounding the first annular region, and that second annular region may contain a liquid and may be termed a shower. In such a situation, the fiber as it leaves the spinneret exit may be surrounded by the shower liquid for the short period of time before the fiber moves into and becomes immersed in the coagulation bath. So, in this situation, the fiber that has left the spinneret might not actually be exposed to air but rather may be surrounded by the shower and later become immersed in the coagulation bath. Nevertheless, the term air gap is still used here for sake of correspondence to other literature in the field of fiber spinning. The shower appears to be important for the manufacture of the outside-in fiber because water in the shower acts as a non-solvent that promotes the formation of the outer selective skin layer. It may be that the combination of the shower and the coagulation bath is important in forming the desired selective (skin) layer on the outside of the fiber. While the emerging fiber is passing through the air gap, it may experience the full magnitude of gravity. This is different from the situation when the fiber is immersed in the coagulation bath, where the fiber is at least partially supported by the buoyant effect of the surrounding coagulation bath liquid. The distance between the spinneret and the coagulation bath may be important to the formation of our skin layer.

The substances that are in contact with any surface of the fiber during the spinning process may influence the transient physicochemical processes that occur. In the bore there may be a bore liquid which serves partly as a place-holding fluid, which also may be chosen to appropriately influence phase separation phenomena in the annular wall, especially in the lumen-facing or internal region of the annular shaped wall of the fiber, after the extruded or spun material emerges from the spinneret exit. If a shower is present, the shower substance may be a composition chosen to appropriately influence phase separation phenomena in the annular shaped wall of the fiber, especially the outward-facing or external region of the annular shaped wall of the fiber, during the short period of time after the extruded or spun material emerges from the spinneret exit before it enters the coagulation bath. The coagulation bath composition may be chosen to appropriately influence phase separation phenomena in the annular shaped wall of the fiber, especially the outward-facing or external region of the wall, during the time when the extruded or spun material is submerged in the coagulation bath. It is believed that, in general, the presence or significant concentration of non-solvent such as water in the compositions adjacent to a surface of the dope promotes quick phase separation or the formation of the skin layer at or near that contact surface, and the presence or significant concentration of organic solvent in a composition adjacent to a surface of the dope slows phase separation at or near that contact surface. In embodiments, the composition of the coagulation bath typically contains the non-solvent and in these experiments the coagulation bath is water. However, it is alternatively possible that some organic solvent could be mixed with water so as to influence the sieving coefficient and molecular weight cutoff of the selective skin layer. For example, it is possible that some concentration (such as about 10 to 20% or even up to 50%) of NMP or similar solvents in either the shower or the coagulation bath or both could be used to further tailor the permeability and sieving properties of the selective skin layer on the outside surface of the fiber. It is believed that the practice in manufacturing conventional Inside-Out fibers uses an air gap in which the fiber actually is exposed to air (or gas), and that there is a larger concentration of non-solvent in the bore liquid.

After the fiber emerges from the spinneret and passes through the coagulation bath, the spun or extruded fiber may be collected on a take-up wheel. The speed of the take-up wheel may be such as to stretch the fiber while the fiber is traveling between the spinneret exit and the take-up wheel. Stretching is likely to change other fiber dimensions in addition to changing length of the fiber, as discussed elsewhere herein.

All of these parameters have an effect on the resulting quantity and dimensions and morphology of the pores in the outer selective layer. In general, the composition of the bore liquid, such as its non-solvent content, and the composition of the shower such as its non-solvent content, and the composition of the coagulation bath such as its non-solvent content, provide parameters that can be varied to influence the phase separation process and the morphology of the fiber.

It is believed that in order to achieve a membrane that maintains the albumin content of the blood, one feature that is desirable is to have a selective layer having pores that are of appropriately small dimension in order to prevent molecules such as albumin from crossing the membrane so that these molecules remain in the blood exiting the cartridge using these fibers.

It is believed that in order to achieve a membrane that has appropriate selectivity, pores of a certain small size (such as several nanometers) and a certain density of such pores (pores per unit volume of local wall region) may be desired to in order provide certain retention or sieving coefficients for certain substances. For example, an effective pore size of 5 nanometers may provide a desirably small albumin sieving coefficient. It is furthermore believed that in order to achieve high flux or high permeability, it is helpful for the selective layer to be thin, in its absolute dimensions. For example, the selective layer may be less than 1 micron, or less than 0.5 micron. Also, this selective layer should retain its integrity and should not excessively crack during manufacture. Small-dimension pores inherently have a high hydraulic resistance, so having a selective layer that is as thin as possible (consistent with structural integrity) will minimize the distance that fluid has to flow through high resistance, and hence will minimize the flow resistance and the transmembrane pressure required for transport.

In order to achieve the desired distribution and morphology of porosity as a function of position within the wall cross-section, it is believed to be desirable to find a suitable combination of polymer dope concentration in combination with a suitable composition and concentration of solvent in the bore liquid (which would help to slow down the phase separation), and a suitable composition of the shower if used, and a suitable bath composition comprising non-solvent. Suitable values of other parameters in the spinning process also can be chosen, such as temperature and take-up speed and spinneret dimensions. What is desired for creating a selective layer may be smaller pores concentrated in a thin layer, and larger pores in other places. Achieving these various goals simultaneously can be challenging.

It is useful to describe here some simultaneous processes occurring during spinning, and how they differ between outside-in fiber spinning and inside-out fiber spinning. In the manufacture of fibers for conventional inside-out dialysis, it is typical to create the selective layer on the inside (lumen) surface of the fiber. This typically is done by co-extruding a dope and a bore liquid, with the bore liquid being a non-solvent such as water or a solution containing a high concentration of water. The non-solvent accelerates the separation of phases on the luminal surface and results in a selective layer having small pores typically tightly packed.

At the same time as the phase separation is happening, the fiber is typically being stretched during the spinning process. The stretching can be approximated as a process that conserves the volume of the material that is being stretched, i.e., conserves the product of length times cross-sectional area of a local region of the fiber. For example, under this assumption, stretching the length by a factor of 4 would be associated with the cross-sectional area of the fiber being reduced by a factor of 4, or the outside diameter of the fiber being reduced by a factor of 2. In the case of an annular dope and a bore liquid in the central region of the annulus, it is possible for this purpose to consider their combined volume as representing the emerging fiber. This is so because during a continuous spinning process there is essentially no opportunity for macroscopic lengthwise flow of one of those substances relative to the other. In the case of the conventional inside-out configuration for dialysis and the conventional fiber geometry, and assuming that the luminal surface solidifies relatively quickly due to the action of the non-solvent while the outside remains relatively softer for a longer duration, this means that the outside surface of the fiber is relatively free to shrink inward as the fiber length increases due to stretching.

In embodiments of the invention, with the goal of creating a fiber for outside-in filtration, the hollow fiber is extruded so as to contain a dope in an annular geometry and a bore liquid occupying the interior of the annulus, but the external surface of the emerging fiber is exposed to a non-solvent more so than is the fiber internal surface (luminal surface). This produces a selective layer on the external surface of the fiber. With a similar assumption that the outside surface of the annular shaped fiber solidifies relatively early in the spinning process, while the interior part of the fiber wall remains softer, and assuming that stretching of the fiber occurs simultaneously with the phase separation or hardening processes, it can be understood that the stretching of such a fiber has the potential to cause a radial or diametral shrinkage of the fiber, trying to pull inward or shrink the outer skin of the in-progress fiber. If the fiber outer surface is already relatively hard when this is trying to happen, this inward pull has the potential to cause the outer skin to buckle into a noncircular shape or to cause the more-interior portion of the wall to separate away from the external skin layer of the fiber. Therefore, the outside-in configuration, with its selective layer on the outside, results in a potential difficulty that is not present, or not nearly as prominent, in the spinning of conventional inside-out fibers.

### Experimental Investigation

Embodiments of the invention are further described through the performance of a series of experiments.

Various fibers were manufactured using a triple concentric spinneret as shown in FIG. 3. FIG. 4 shows the overall arrangement of the spinneret, pumps, baths and take-up wheel.

In the course of experimental work for embodiments of the invention, fiber spinning was performed to produce experimental fibers under 16 different sets of conditions. These are designated as fibers F1 through F16. For all of the fibers, certain basic measurements were taken, such as measurement of dimensions, and photographic documentation, and observation of overall appearance. A subset of those fibers was further characterized to measure additional properties such as flow resistance or passage of certain molecular weight solutes or mechanical properties.

The experimental conditions for the series of experiments are described in Table 1.

TABLE 1

| | Dope | Bore | | | | | | | | | | |
|---|---|---|---|---|---|---|---|---|---|---|---|---|
| | (PES/ | liquid | Show-er | Bath | | | Dope + | | | Take-up | | |
| | PVP/ | (NMP/ | liquid % | liquid % | Dope flow | Bore flow | Bore | Shower | | wheel | Nozzle | Bath |
| Fiber | NMP) | water) | Water | Water | rate | rate | flow rate | flow rate | Air gap | speed | temp | temp |
| Units | Wt % | Wt % | Wt % | Wt % | mL/min | mL/min | mL/min | mL/min | cm | m/min | C. | C. |
| F1 | 15/7/78 | 50/50 | 100 | 100 | 1 | 0.9 | 1.9 | 0.3 | 0.6 | 9 | RT | RT |
| F2 | 15/7/78 | 50/50 | 100 | 100 | 1 | 0.4 | 1.4 | 0.3 | 0.6 | 9 | RT | RT |
| F3 | 15/7/78 | 75/25 | 100 | 100 | 1 | 0.4 | 1.4 | 0.3 | 0.6 | 9 | RT | RT |
| F4 | 15/7/78 | 90/10 | 100 | 100 | 1 | 0.4 | 1.4 | 0.3 | 0.6 | 9 | RT | RT |
| F5 | 15/7/78 | 75/25 | 100 | 100 | 0.6 | 0.4 | 1 | 0.3 | 0.6 | 9 | RT | RT |
| F6 | 15/7/78 | 75/25 | 100 | 100 | 0.4 | 0.4 | 0.8 | 0.3 | 0.6 | 9 | RT | RT |
| F7 | 15/7/78 | 75/25 | 100 | 100 | 0.4 | 0.2 | 0.6 | 0.3 | 0.6 | 9 | RT | RT |
| F8 | 15/7/78 | 75/25 | 100 | 100 | 0.4 | 0.1 | 0.5 | 0.3 | 0.6 | 9 | RT | RT |
| F9 | 15/7/78 | 75/25 | 100 | 100 | 0.35 | 0.1 | 0.45 | 0.3 | 0.6 | 9 | RT | RT |
| F10 | 15/7/78 | 75/25 | 100 | 100 | 0.3 | 0.1 | 0.4 | 0.3 | 0.6 | 9 | RT | RT |
| F11 | 15/7/78 | 75/25 | 100 | 100 | 0.25 | 0.1 | 0.35 | 0.3 | 0.6 | 9 | RT | RT |
| F12 | 15/7/78 | 75/25 | 100 | 100 | 0.2 | 0.2 | 0.4 | 0.3 | 0.6 | 14 | RT | RT |
| F13 | 15/7/78 | 75/25 | 100 | 100 | 0.2 | 0.2 | 0.4 | 0.3 | 0.6 | 18 | RT | RT |
| F14 | 12/5.6/82.4 | 75/25 | 100 | 100 | 0.4 | 0.1 | 0.5 | 0.3 | 0.6 | 9 | RT | RT |
| F15 | 12/5.6/82.4 | 75/25 | 100 | 100 | 0.4 | 0.2 | 0.6 | 0.3 | 0.6 | 9 | RT | RT |
| F16 | 12/5.6/82.4 | 75/25 | 100 | 100 | 0.5 | 0.2 | 0.7 | 0.3 | 1.2 | 9 | RT | RT |

Note:
RT = Room Temperature (approximately 20° C.)

The hollow fibers were produced by non-solvent induced phase separation. The polymer dope solutions were prepared by dissolving Ultrason E6020 PIES (BASF, Ludwigshafen, Germany) (polyethersulfone) (Molecular Weight=75000 g/mol. Polydispersity index (dispersity) (Mw/Mn)=3.4) and PVP K90 (molecular weight=360 kDa, Sigma-Aldrich Chemie GmbH, Munchen, Germany) (polyvinylpyrrolidone) in ultrapure N-methyl pyrrolidone (NMP) (Acros Organics, Geel, Belgium). All dope polymer solutions (Table 1) were mixed on a roller bench for three days, and then they were filtered using a Bekipor ST AL315 μm filter (Bekaert, Kortrijk, Belgium). They were transferred into stainless-steel syringes and left to degas for at least 24 hours. After this, a syringe containing the dope solution was connected to a high-pressure syringe pump and to the spinneret for the manufacture of the fiber. Ultrapure water was used as the shower liquid, and it was pumped through the spinneret at a flowrate of 0.3 mL/min. The shower flow rate for all the experiments was set at 0.3 mL/min. The term “shower” refers to the external coagulant liquid pumped through the outermost annular orifice of the spinneret (FIG. 3). The bore liquid was a mixture of ultrapure water and NMP made at various concentrations as described in Table 1. The coagulation bath consisted of demineralized water at room temperature, about 20° C. After spinning, the fabricated hollow fibers were washed several times with demineralized water to remove any remaining solvent and then they were stored for further use.

All fibers reported here were spun using a shower flow rate of 0.3 mL/min. This flow rate was selected because it enabled production of a regular dense outside layer without morphological irregularities. This is an important parameter for making the outside-in fiber. In the entire set of development work, including experiments that were not the fibers F1-F16 reported here, the following shower flowrates were used: 0.04 mL/min; 0.3 mL/min; 0.6 mL/min; 1.2 mL/min; 2.4 mL/min; 4.8 mL/min. With higher shower flow rates, the morphology of the fibers was quite irregular and delamination of the outer layer was observed, while with lower shower flow rates it was not possible to obtain a desired dense outer layer and delamination of the outer layer was also observed. Therefore, for all of the fibers F1-F16, the shower flowrate used was 0.3 mL/min.

The coagulation bath consisted of demineralized water at room temperature, approximately 20° C., which was the same temperature as other fiber spinning fluids and the equipment itself. The effects of many parameters of the spinning procedure (i. e. bore liquid composition, dope composition, dope and bore flow rates, take-up wheel speed, air gap length and polymer dope concentration) were investigated in various combinations.

Although in these experiments the properties of a non-solvent are provided for the shower and the coagulation bath and the collecting bath by using pure water for all of those, it is alternatively possible that any of those fluids could have a small concentration of organic solvent along with a large concentration of water, and would still have substantial non-solvent properties. The organic solvent could be n-methyl pyrrolidone as was used in the dope, or some other organic solvent. Any such variation could be used to achieve specific properties of the outer selective layer. For example, the shower and the coagulation bath and the collecting bath could have less than a 1% concentration of organic solvent or less than a 2% concentration of organic solvent that is a solvent for the polymeric materials. Non-solvent refers to a substance in which the polymeric materials that are contained in the dope are not significantly soluble. Water is one example. Other examples include isopropanol, glycerol, and mixtures of any of these non-solvent substances.

The air gap between the spinneret and the coagulation bath was adjusted to be either 0.6 cm or 1.2 cm depending which experiment was being performed (also described in Table 1). A take-up wheel was used for the collection of the fibers after they left the spinneret. Various different flow rates of the dope and bore liquids and various different speeds of the take-up wheel were used for the fabrication of various fibers, as described in Table 1.

In general, all of the feed solutions and the baths and the spinneret were at identical temperatures, which was room temperature, approximately 20° C. The various temperatures could be essentially identical to each other, or within 2° C. of each other, or within 5° C. of each other, or within 10° C. of each other. The various temperatures may be approximately 20° C., or approximately 18-22° C., or approximately 20-25° C., or approximately 20-30° C., or other values in similar ranges. If desired, it would alternatively be possible to perform fiber manufacturing using other temperatures, either all of the feed solutions and baths and the spinneret being of identical temperatures at some other temperature, or with the various feed solutions and baths and spinneret being at temperatures different from each other.

### Measurement Techniques and Protocols

For the fibers described in these Examples, the morphology was analyzed by a Scanning Electron Microscope (SEM) (JEOL JSM-IT 100, Tokyo, Japan). For the imaging of the cross-sections, the membranes were dried in air and fractured in liquid nitrogen. Prior to SEM imaging, the samples were gold sputtered using the Cressington 108 auto sputter (Cressington Scientific Instruments, Watford, UK).

Analysis of the outer surface chemistry of selected fibers was performed by Attenuated Reflectance—Fourier Transmittance Infrared (ATR-FTIR) spectroscopy (Spectrum Two, PerkinElmer) and Spectrum Quant software. All scans were performed at room temperature in triplicate on various parts of the membrane surface at a resolution of 4 cm⁻¹ and were compared to FTIR scans of pure PES, pure PVP materials and to the commercial hollow fiber Fresenius F8HPS.

Analysis of selected fibers was performed using X-ray photoelectron spectroscopy (XPS) using a Quantera scanning XPS microprobe (Physical Electronics, Chanhassen, Minn., USA) with Al Kα excitation radiation (hv=1486.6 eV). The given elemental atomic percentages were measured for fiber F16 and, for comparison, were also measured for a Fresenius commercial fiber F8HPS. Data analysis was performed using Compass for XPS control, Multipak v 9.4.0.7.

For determining the properties of passage of solutes through the membrane as a function of Molecular Weight, experiments were conducted using several different test liquids containing substances having respective Molecular Weights. The Molecular Weight Cut Off was characterized to the extent that experiments with these several fluids having varied Molecular Weights can provide data points at the respective Molecular Weights so as to roughly describe the cutoff characteristics. The transport characteristics of the fibers were experimentally measured using pure water, model albumin and vitamin B12 aqueous solutions, as well as creatinine in human blood plasma fluid. These tests were performed only for a small subset of the fibers.

For water transport/permeability experiments, selected fibers were dried in air and membrane modules with known surface areas (fiber F3, n=3, 4.1±0.8 cm²; fiber F4, n=2, 4.1±0.0 cm²; fiber F8, n=2, 2.60.0 cm²; fiber F15, n=3, 2.8±0.1 cm²; fiber F16, n=3, 4.8±2.9 cm²) were prepared using a two-component epoxy glue (Griffon Combi Snel-Rapide, Bison International, Goes, The Netherlands). Before the water transport experiments, the fiber modules were pre-wetted with ethanol for 30 minutes at a transmembrane pressure (TMP) of 1 Bar and were pre-compacted with ultra-pure water at a TMP of 1 Bar for at least 30 min. Afterwards, the amount of permeated water was measured over time at TMP of 0.6, 0.8 and 1 Bar. The resulting water permeability was calculated as the slope of the linear fit of the flux (L/(m² h)) versus the TMP (in Bar). In addition to providing a measurement of the transport property, this experimental procedure was used to demonstrate that the fibers are mechanically strong and can tolerate a pressure difference of 1 bar.

Albumin filtration experiments were also performed using membrane modules. The membrane modules were prepared using a two-component epoxy glue and had known effective membrane surface area. They were prepared using several different fibers that had been developed herein (fiber F3, n=2, 3.6±0.6 m2; fiber F4, n=2, 4.1±0.0 m2; fiber F8, n=2, 2.60.0 m2; fiber F15, n=3, 2.80.1 m2; fiber F16, n=2, 5.8±3.1 m2) in dead-end configuration. The modules were pre-wet with ethanol at 1 Bar for at least 30 minutes and pre-pressurized with water at 1 Bar for at least 30 minutes. For these filtration experiments, bovine serum albumin (BSA) (66.5 KDa) was used (Sigma-Aldrich Chemie GmbH, Munchen, Germany). BSA solution at a concentration of 0.6±0.0 g/L in Phosphate Buffer Saline (PBS) at pH 7.4 was pressurized in the outside-in configuration from the outside compartment to the lumen compartment of the fibers at a pressure of 1 Bar. After 30 minutes the permeate was collected and albumin concentration in the permeate was measured using a UV spectrophotometer (NanoDrop Technologies, Wilmington, Del.). The sieving coefficient (SC) was calculated using the equation given elsewhere herein.

Vitamin B12 filtration experiments were performed on membrane modules of the outside-in fiber designated F16 (n=3, 9.9±0.3 m2) in dead-end configuration. The modules were pre-wet with ethanol at 1 Bar for at least 30 minutes and pre-pressurized with water at 1 Bar for at least 30 minutes. Vitamin B12 solution at a concentration of 0.1 g/L in Phosphate Buffered Saline (pH 7.4) was pressurized in the outside-in configuration from the dialysate compartment to the lumen compartment of the fibers at a pressure of 1 Bar. After 30 minutes the permeate was collected and the concentration of Vitamin B12 was measured using a UV spectrophotometer (NanoDrop Technologies, Wilmington, Del.). The sieving coefficient was calculated using the equation given elsewhere herein.

The removal of creatinine from human plasma by the outside-in fiber designated F16 was investigated in the diffusion mode (Transmembrane pressure (TMP)=0) and in a counter-current configuration using a dedicated set-up (Convergence, Enschede, The Netherlands). 50 mL of human plasma (obtained from healthy donors in compliance with local ethical guidelines—Sanquin, Amsterdam, The Netherlands) was spiked with creatinine (Sigma-Aldrich Chemie GmbH, Schnelldorf, Germany) (0.1 g/L) and was recirculated at a flow rate of 10 mL/min in the space outside the fibers. 50 mL of dialysis fluid was recirculated at a flow rate of 1 mL/min in the intraluminal space. To prepare the dialysis fluid, 2 mM KCl, 140 mM NaCl, 1.5 mM CaCl₂), 0.25 mM MgCl₂, 35 mM NaHCO₃ (all from Sigma-Aldrich Chemie GmbH, Schnelldorf, Germany) and 5.5 mM glucose (Life Technologies Europe BV, Bleiswijk, The Netherlands) were dissolved in water prepared using a Milli-Q water purification system. Membrane modules composed of 3 fibers with a total outer surface area of 2.9±0.1 cm² were used. The experiments were performed in triplicate for 24 hours and samples were taken every hour for the first 4 hours and at 24 hours from the blood plasma and dialysate compartments for quantification of creatinine. Creatinine concentrations were analyzed by UV detection using reverse-phase high-performance liquid chromatography (RP-HPLC). Creatinine concentrations were analyzed both in plasma (after filtration through 30 kDa filters, Amicon Ultracel-30 K, Merck Millipore Ltd) and in the dialysate. Creatinine diffusive removal was calculated by the creatinine concentration found in the dialysate. All removal results were normalized to the outer surface areas of the fiber modules.

Mechanical tests of the experimental fiber F16 and the commercial fiber F8HPS (Fresenius) hollow fiber were performed using a Zwick Z020 tensile tester equipped with a 500 N load cell at room temperature. Ultimate tensile strength, Young's Modulus and elongation at ultimate strength were obtained.

All the data are presented as mean±SD (standard deviation). Statistical analyses were performed using GraphPad Prism version 5.02 (GraphPad Prism Software, La Jolla, Calif., USA). Statistical differences for the mechanical test were determined using unpaired Student's t-test. Multiple comparisons between different groups were performed using one-way analysis of variance (ANOVA) with Bonferroni post-hoc test in order to determine statistical differences of the thicknesses of the selective layers of the developed fibers. Differences were considered significant at p<0.05.

For some analysis and comparison, a commercially available fiber F8HPS (Fresenius) was used for comparison of the surface chemistry and mechanical properties.

### Experimental Results

The following non-limiting Examples present various forms of further analysis of this information.

### Example 1

FIGS. 5-8 present Scanning Electron Microscope (SEM) images of all the fibers that were produced. For each fiber, an image is presented of the cross-section, a magnification of the outer region, and a magnification of the inner (lumen) region. From these, first, some general observations can be made based on visual appearance.

In general, a fiber of an embodiment of the invention may comprise a selective layer on the outside and a supporting porous region that is located more interiorly. For present purposes, it is desirable that the fiber have a selective skin layer on the exterior surface. All of the fibers illustrated have that feature but there are differences among the various fibers based on the conditions used to make them. Some of the fibers also have a dense layer on the interior surface, which might not be desirable for the present application. In some fibers the porous supporting region is spongy and approximately isotropic. In other fibers, the porous supporting region contains elongated macrovoids, which are generally radially oriented. In some of the fibers, there is delamination within the wall of the hollow fiber. In some fibers, the exterior of the fiber has a noncircular irregular shape such as a polygon.

All of this data is summarized in Table 2, which is an extension of Table 1. Table 2 presents, for all 16 fibers, additional information in the form of various measurements of the manufactured fibers.

TABLE 2

| | | | | | | | Fiber | Fiber | |
|---|---|---|---|---|---|---|---|---|---|
| | Bore | Dope | Shower | | | | Outside | Inside | Wall |
| Fiber | area | area | area | Stretch | Qdope/ | Speed | Diameter | Diameter | Thickness |
| Units | mm\hat{ }2 | mm\hat{ }2 | mm\hat{ }2 | Ratio | Qshower | Ratio | μm | μm | μm |
| F1 | 0.031 | 0.165 | 0.188 | 0.93 | 3.33 | 3.81 | 643 | 564 | 43 |
| F2 | 0.031 | 0.165 | 0.188 | 1.26 | 3.33 | 3.81 | 448 | 368 | 41 |
| F3 | 0.031 | 0.165 | 0.188 | 1.26 | 3.33 | 3.81 | 534 | 398 | 70 |
| F4 | 0.031 | 0.165 | 0.188 | 1.26 | 3.33 | 3.81 | 503 | 390 | 50 |
| F5 | 0.031 | 0.165 | 0.188 | 1.77 | 2.00 | 2.29 | 497 | 364 | 65 |
| F6 | 0.031 | 0.165 | 0.188 | 2.21 | 1.33 | 1.52 | 437 | 369 | 34 |
| F7 | 0.031 | 0.165 | 0.188 | 2.95 | 1.33 | 1.52 | 370 | 276 | 49 |
| F8 | 0.031 | 0.165 | 0.188 | 3.53 | 1.33 | 1.52 | 330 | 191 | 72 |
| F9 | 0.031 | 0.165 | 0.188 | 3.93 | 1.17 | 1.33 | 310 | 190 | 61 |
| F10 | 0.031 | 0.165 | 0.188 | 4.42 | 1.00 | 1.14 | 294 | 188 | 47 |
| F11 | 0.031 | 0.165 | 0.188 | 5.05 | 0.83 | 0.95 | 272 | 166 | 55 |
| F12 | 0.031 | 0.165 | 0.188 | 6.87 | 0.67 | 0.76 | 297 | 196 | 50 |
| F13 | 0.031 | 0.165 | 0.188 | 8.84 | 0.67 | 0.76 | 252 | 124 | 67 |
| F14 | 0.031 | 0.165 | 0.188 | 3.53 | 1.33 | 1.52 | 317 | 201 | 52 |
| F15 | 0.031 | 0.165 | 0.188 | 2.95 | 1.33 | 1.52 | 338 | 243 | 48 |
| F16 | 0.031 | 0.165 | 0.188 | 2.52 | 1.67 | 1.90 | 322 | 236 | 42 |

| | | | | | Thickness | KUf | Prtmea- | |
|---|---|---|---|---|---|---|---|---|
| | | | | | of | (mL/(h · | bility | |
| | Aspect | | | Finger- | selective | mmHg · | (mL/(h · | |
| Fiber | ratio | Dense | | like | layer | )) for | mmHg · | |
| Units | OD/ID | layer | Morphology | macro-voids | μm | for2 m2 | m2)) | SC, BSA |
| F1 | 1.1401 | Inner and outer | Good | Spongy | 2.4 ± 0.3 | | | |
| F2 | 1.2174 | Inner and outer | Good | Spongy | 2.6 ± 0.6 | | | |
| F3 | 1.3417 | Outer | Good | Spongy | 6.9 ± 0.2 | 19.6 ± 0.2 | 9.8 ± 0.1 | 0.00 ± 0.00 |
| F4 | 1.2897 | Outer | Good | Spongy | 2.1 ± 0.0 | 476 ± 90 | 238 ± 45 | 0.98 ± 0.00 |
| F5 | 1.3654 | Outer | | Partial | 1.4 ± 0.0 | | | |
| F6 | 1.1843 | Outer | | Spongy | 1.3 ± 0.4 | | | |
| F7 | 1.3406 | Outer | | Spongy | 1.3 ± 0.0 | | | |
| F8 | 1.7277 | Outer | Good | Partial | 1.5 ± 0.1 | 22.6 ± 2.6 | 11.3 ± 1.3 | 0.02 ± 0.01 |
| F9 | 1.6316 | Outer | Irregular + | Yes | | | | |
| | | | delaminated | | | | | |
| F10 | 1.5638 | Outer | Irregular + | Yes | | | | |
| | | | delaminated | | | | | |
| F11 | 1.6386 | Outer | Irregular + | Partial | | | | |
| | | | delaminated | | | | | |
| F12 | 1.5153 | Outer | Irregular + | Partial | | | | |
| | | | delaminated | | | | | |
| F13 | 2.0323 | Outer | Irregular + | Partial | | | | |
| | | | delaminated | | | | | |
| F14 | 1.5771 | Outer | Irregular | Yes | 0.9 ± 0.0 | 62.2 ± 4.8 | 31.1 ± 02.4 | |
| F15 | 1.3909 | Outer | Good | Yes | 0.7 ± 0.1 | 25.1 ± 2.9 | 12.5 ± 1.4 | 0.02 ± 0.00 |
| F16 | 1.3644 | Outer | Good | Yes | 0.6 ± 0.1 | 31.6 ± 6.4 | 15.8 ± 3.2 | 0.09 ± 0.01 |

### Example 2: Fiber Outside Diameter, as a Function of Dope Flowrate

A basic dimensional parameter of interest is the outside diameter of the fiber, because a dialysis fiber should have an outside diameter of about 200 to 300 microns in order to be suitable to make an acceptable dialyzer. It can be expected that in general this parameter is a function at least of the dope flowrate through the bore channel of the spinneret. The dope contains the polymeric material (a mixture of PIES and PVP) that ultimately becomes the wall of the hollow fiber. Accordingly, FIG. 9 is a plot, for all of the fibers produced, of the measured fiber outside diameter plotted as a function of the dope flowrate.

FIG. 9 shows the data for all 16 of the produced fibers, which means that the data includes variations in several of the other manufacturing parameters, which were varied in various ways and combinations during the 16 experiments. Therefore, the plot in FIG. 9 should not be expected to indicate perfect correlation; rather, FIG. 9 could be expected to provide a general indication or correlation about the outside diameter. FIG. 9 indicates that generally, a larger dope flowrate roughly correlates with a larger fiber outside diameter. A fiber outside diameter of >350 microns is undesirable for present applications. The fibers that had Outside Diameter >350 microns all had a dope flowrate of at least 0.4 ml/min. The fibers that had smaller Outside Diameter mostly had smaller dope flowrates. (This correlation does not take into account the stretching of the fiber after the fiber leaves the spinneret. This stretching, due to the speed of the take-up wheel, influences the outside diameter, and the speed of the take-up wheel varied among different values during some of the experiments reported here.)

In an attempt to produce fibers having especially small outside diameters, the dope flow rate was further reduced in a stepwise manner for fibers F8-F11 (with the dope flowrates being 0.4 mL/min, 0.35 mL/min, 0.3 mL/min and 0.25 mL/min, respectively) at a fixed bore flow rate of 0.1 mL/min. Even though a decrease of various fiber dimensions did occur (both wall thickness and inner diameter were reduced), it was found that the morphology of fibers F9, F10 and F11 is irregular, there can be observed delamination of the inner/lumen layer, and it can be observed that the inner and outer circumference are non-circular (FIG. 4, Table 2). The results show that, with this spinneret, when using a very low bore flow rate of 0.1 mL/min, the smallest dope flow rate that can be used to produce fibers having a regular external shape is 0.4 mL/min.

### Example 3: Fiber Outside Diameter, as a Function of Combined Flowrate of Dope and Bore Liquid

It can further be thought that in an overall sense, what makes up the volume of the fiber as the fiber progresses through the stages of the manufacturing process is the volume of dope together with the volume of bore liquid that is contained within the annularly-shaped dope region. Accordingly, it might be considered that the fiber outside diameter could correlate with the total flowrate of dope and bore in combination. Therefore, FIG. 10 shows a plot of the fiber outside diameter as a function of the total flowrate of dope and bore solution added together. Just as in FIG. 9, the data in FIG. 10 includes variations in several of the other manufacturing parameters, which were varied in various ways and combinations during the 16 experiments. It can also be kept in mind that neither FIG. 9 nor FIG. 10 takes into account the stretching of the fiber during the latter part of the manufacturing process, which sometimes varied among the various experiments. Therefore, again, it should not be expected that there would be a perfect correlation. However, it is believed that FIG. 10 shows a better correlation between the plotted flowrate and the fiber outside diameter, compared to FIG. 9.

A rationale for considering the total flowrate of bore liquid and dope in combination can further be found by noting the phenomenon of die swell. In general, during spinning, the bore liquid volumetric flowrate and the dope volumetric flowrate can be separately controlled or imposed, because each is pumped through a dedicated separately controllable positive displacement pump. The bore liquid leaves the spinneret at a bore liquid linear velocity that can be represented as the bore liquid volumetric flowrate divided by the area of the innermost circular discharge. Similarly, the dope leaves the spinneret at a dope linear velocity that can be represented as the dope volumetric flowrate divided by the area of the annular region through which the dope flows. Because of the independent control of the two flowrates, it is possible that the bore liquid linear velocity and the dope linear velocity at the spinneret exit may be different from each other. It could, however, be physically expected that the two velocities will quickly equilibrate with each other. There is essentially no opportunity for relative lengthwise flow of one fluid relative to the other along the length of the fiber, because the fiber-spinning process is a continuous process. In general, the bore liquid typically is a liquid that is fairly low-viscosity and Newtonian. Typically for manufacturing conventional inside-out fibers the bore liquid is mostly water, and for embodiments of the invention the bore liquid is mostly organic solvent. Water is a low-viscosity Newtonian fluid, and typical organic solvents such as n-methyl pyrrolidone have properties that are generally similar to the properties of water (viscosity in the range of less than 10 mPa-s). Thus, based on conservation of volume of the bore liquid, it could be expected that the bore would not change its cross-sectional area or dimension (diameter) very much unless the entire fiber is stretched longitudinally (which is discussed elsewhere herein). For the dope, the situation may be somewhat different. The dope, which is a solution of polymer(s) in an organic solvent, typically has a significantly larger viscosity than water and also has viscoelastic properties. The process of extruding a fiber of such dope is known to exhibit the phenomenon of die swell, in which the material upon exiting the die expands somewhat in its cross-section, because of its elastic properties. If there is a mismatch between the nominal bore liquid linear velocity and the nominal dope linear velocity, this phenomenon would provide an opportunity for the exiting polymer to better match the bore liquid linear velocity by adjusting its outward die swell expansion so that the resulting dope velocity better matches the bore liquid linear velocity. If necessary to achieve matching of local velocity between the bore liquid and the dope, the dope can undergo die swell partly inward and partly outward. The relative amounts of inwardly-directed die swell and outwardly-directed die swell can self-adjust as needed for particular fiber-spinning conditions.

So far, none of the discussion in this Example considers the phenomenon of stretching as is imposed by the take-up wheel, which is considered starting in the next Example. The stretching provides a further opportunity for the annularly shaped dope to adjust its dimensions and velocity, while being stretched, so as to equalize the bore liquid linear velocity after stretching and the bore liquid diameter after stretching.

### Example 4: Effect of Take-Up Wheel Speed

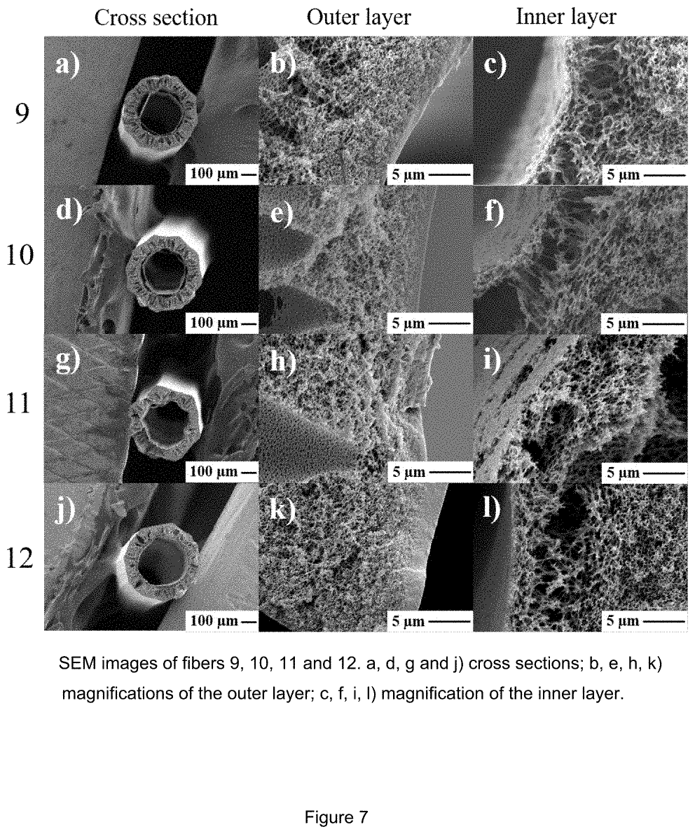

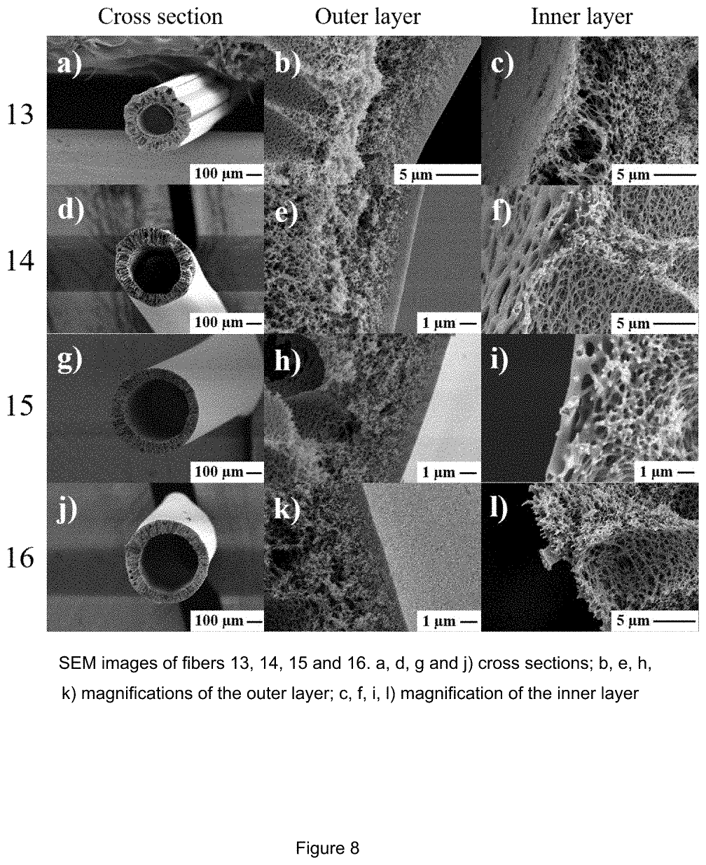

In general, during the set of experiments described herein, the take-up wheel speed was such as to stretch the fiber and reduce its cross-sectional dimensions during passage of the fiber between the spinneret exit and the take-up wheel. The factor of stretching ranged from slightly more than 1 (which represents hardly any stretching) to as large as approximately 9. It is expected that, due to the pulling and stretching action, faster take-up speeds would decrease the overall dimensions of the fibers in cross-section. The take-up wheel speed was varied in some of the experiments. Most of the fibers produced in this work were spun using a take-up wheel speed of 9±1 m/min, except that fibers F12 and F13 were spun with take-up wheel speeds of 14±1 m/min and 18±1 m/min, respectively. For the two fibers that were spun with larger take-up speeds than the rest of the fibers, the increased take-up speed resulted, as expected, in overall decrease of the fiber dimensions. However, those two fibers, F12 and F13, have irregular morphology characterized by delamination of the interior from the outer skin layer, and they also have outer and inner circumferences that are irregular in shape (FIG. 7, FIG. 8, Table 2). There were also several other fibers, fibers F9-F11, which were spun using the more usual take-up wheel speed, which also displayed irregular morphology and delamination.

### Example 5: Volumetric Calculations, in Regard to Fiber Outside Diameter, that Include Take-Up Wheel Speed

It is furthermore possible to perform volumetric-based fiber dimensional calculations as done in some previous examples but also taking into account the speed of the take-up wheel. Essentially, it is possible to calculate the total volume of extruded dope flow plus bore liquid flow, in a given amount of time, and divide that amount by the linear distance of the take-up wheel in the same given amount of time. This results in a calculated cross-sectional area and hence an outside diameter of the fiber based on consideration of the total amount of the dope and the bore liquid. The calculated outside diameter is plotted in FIG. 11 as the horizontal axis. The vertical axis in FIG. 11 is the measured outside diameter of the same fiber. There is reasonably good correlation. The dashed line shows the theoretically expected 1:1 relation. It can be understood that the data plotted in FIG. 11 still includes variations in several of the other manufacturing parameters, which were varied in various ways and combinations during the 16 experiments. From this correlation, it is possible to approximately predict the diameter of the fiber. This approach can be used to predict or estimate fiber outside diameter in advance of an experiment.

A further consideration is that although the space occupied by the dope is believed to generally correlate with the space that is eventually occupied by the solidified fiber, it is true that the dope actually contains (by weight) more solvent than it contains polymer. The solvent can be expected to eventually evaporate or be rinsed out or disappear in some other manner. It is believed that, generally speaking, the solvent of the dope is replaced by pore space. The bore fluid also evaporates or disappears, leaving behind the lumen as the space formerly occupied by the bore liquid.

### Example 6. Delamination and Irregular Shape Correlated with Stretch Ratio

Among the 16 experimental fibers, Fibers F9 through F13 exhibited delamination and an irregular external shape, while the other fibers exhibited a normal condition and external shape. In order to correlate this, a Stretch Ratio has been calculated for the manufacturing conditions for each fiber. This is illustrated in FIG. 12.

Calculation of the stretch ratio begins with calculating a linear velocity of the fluids exiting the spinneret that are located within the envelope of the eventual fiber. Therefore, this calculation is performed on the basis of the total of the dope flowrate plus the bore flowrate, because at the time of exit from the spinneret and early formation of the fiber, both of these liquids are contained inside the boundary of what will eventually be the fiber. From a point of view of the immediate exit from the spinneret, it is possible to calculate an actual linear velocity of the bore liquid as the volumetric flowrate of the bore liquid divided by the area of the bore region of the spinneret. Similarly, it is possible to calculate an actual linear velocity of the dope as the volumetric flowrate of the dope divided by the cross-sectional area of the annular region of the spinneret through which the dope is dispensed. In general, at the point of exiting from the spinneret exit, the actual linear velocity of the dope and the actual linear velocity of the bore liquid do not have to be equal to each, because for example, in a practical sense, the dope flowrate and the bore liquid flowrate are provided by separate pumps that are independently operable. If these two linear velocities differ from each other, it can be expected that these two velocities would have a tendency to equalize with each other because of the intimate contact between the dope and the bore liquid. Also, both the dope and the bore liquid, being fluids, both have the ability to adjust their cross-sectional dimensions to accommodate the situation. It is assumed that there is no relative flow of either liquid along the length direction of the fiber relative to the other liquid, because the spinning process is continuous. It is believed that both the dope and the bore liquid become stretched during spinning (due to the pulling action of the take-up wheel), and it might be expected that the relative amounts of stretching would adjust themselves such that the linear velocity of the bore liquid and the linear velocity of the dope equalize with each other. Therefore, it would be appropriate to consider a representative linear velocity that is calculated using the total flowrate inside the boundary of the fiber, that is, the sum of the dope volumetric flowrate and the bore liquid volumetric flowrate). This total flowrate is divided by the cross-sectional area of the spinneret exit for dope flow, which in this case is a circle having diameter of 500 microns. Then this linear velocity is compared to the take-up wheel velocity. The Stretch Ratio is the take-up wheel linear velocity divided by the fiber linear velocity at the exit of the spinneret.

This Stretch Ratio parameter is further described by the following equations.

Q(bore+dope)=Qbore+Qdope

Area(bore+dope)=Area(bore)+Area(dope)

Exit velocity(bore+dope)=Q(bore+dope)/Area(bore+dope)

TakeupVel=linear velocity of take-up wheel

Stretch Ratio=TakeupVel/Exitvelocity(bore+dope)

The data are plotted in FIG. 12 for all 16 experimental fibers. It can be seen from the experimental results that a Stretch Ratio greater than 4 is consistently associated with delamination and irregular external shape of the finished fiber, while a Stretch Ratio less than 4 is consistently associated with a normal, desirable appearance and condition of the finished fiber. Of course, during the experimentation some other manufacturing variables were also varied in various ways, but it seems that the Stretch Ratio as defined here is a useful discriminator of the likelihood of delamination and irregular cross-sectional shape irrespective of some other manufacturing details. This criterion can be used to select manufacturing conditions that avoid delamination and irregular cross-sectional shape.

It is suggested here, although it is not wished to be limited to this explanation, that the association of the delamination and the irregular cross-sectional shape with situations of large Stretching Ratio may be an effect of conservation of volume, in combination with some solidification dynamics during the spinning process, which can also be thought of as an effect that is similar to Poisson's Ratio. Poisson's Ratio is the negative of the ratio of transverse strain to longitudinal strain. For polymeric materials and many other solids, under elastic deformation conditions, a typical value of Poisson's Ratio is in the range of 0.3 to 0.5. A Poisson's Ratio of 0.5 corresponds to conservation of volume during deformation. Poisson's Ratio is most commonly used in situations of elastic deformation. In the present situation of large plastic deformation and flow, the large plastic deformations and incompressible liquid flow are probably best described as a situation of conservation of volume. In any event, the general conclusion is that if during spinning a fiber is stretched longitudinally to a certain longitudinal strain, the fiber will try to shrink in a transverse direction by a strain amount that approximates 50% of the longitudinal strain. This shrinkage means a reduction of the outside diameter of the fiber as it emerges and forms, and possibly also of its wall thickness.

In embodiments of the invention, the observed delamination and shape phenomena for certain fibers can be understood if, in connection with certain dynamics of the fiber spinning process, it is assumed that the outside skin or selective layer of the fiber forms or solidifies relatively quickly in comparison to the inner layer. In embodiments of the invention, the presence of the non-solvent in the external shower and in the coagulation bath may promote relatively fast phase separation in order to form small-pore structures, because formation of small-pore structures is generally associated with fast processes. In contrast, in embodiments of the invention, the lumen is exposed to a solvent-rich solution that is formulated to keep the inward-facing surface relatively softer for a relatively long period of time in order to allow or promote the formation of pores and other structures that are relatively larger. Then, if the outer skin forms relatively early and the outer perimeter of the fiber is set or hardened fairly early and then the fiber continues to stretch as it progresses through the coagulation bath between the spinneret and the take-up wheel, there can be expected to be a tendency for circumferential shrinkage or relative inward motion of the fiber wall due to conservation of volume in response to the stretching. If the outer skin is already formed or solid or almost so, the outer skin might be unable to participate in radially inward motion and therefore might respond by buckling or by delaminating from interior material or both. During such longitudinal stretching of the fiber, the inner luminal surface is constrained because the lumen is filled with a liquid, namely the bore liquid, which is neither compressible nor expandable and there is no freedom for additional bore liquid to flow in along the lengthwise direction in order to allow radial expansion of the lumen. Thus, the inner (luminal) surface of the hollow fiber can be expected to move radially inward as stretching progresses, thereby attempting to pull the rest of the fiber wall inward also.

This explanation would be consistent with the fact that irregular shape and delamination occurred only at the largest Stretch Ratios. It is furthermore believed that in contrast, during the spinning of conventional Inside-Out fibers having a dense layer on the inside (luminal) surface, the corresponding risk of delamination is not present, specifically because of the differing placement of the selective layer. In those conventional fibers, if the dense layer becomes hard earlier on the inner (luminal) surface than occurs on the outer layer, and then further stretching occurs, the effect of conservation of volume or Poisson's ratio would simply be to reduce the outer diameter of the fiber, and there is no geometric or physical constraint on the outside of the fiber such as to restrain that outer surface against motion in the radial direction. Thus, the exterior of the conventional fiber is free to adjust its dimensions during spinning and stretching. This would explain the problem of delamination being a more significant problem for spinning an Outside-In fiber than is believed to be the case for spinning a conventional Inside-Out fiber.

The results plotted in FIG. 12 show that all of the delaminated/polygonal fibers occurred at a Stretch Ratio above a certain value, and all of the normal fibers occurred at a Stretch Ratio below that value. That value is approximately 4. This pattern is found to be a consistent pattern even while various other manufacturing parameters were varied in various ways among the 16 fiber manufacturing runs. Thus, the Stretch Ratio can be used as a significant guiding parameter in making successful Outside-In fiber. For example, the Stretch Ratio can be kept to a value less than approximately 4, or less than 3. However, it is not wished to be limited to this explanation.

### Example 7: Relation Between Bore Flow Rate and Fiber Inside Diameter

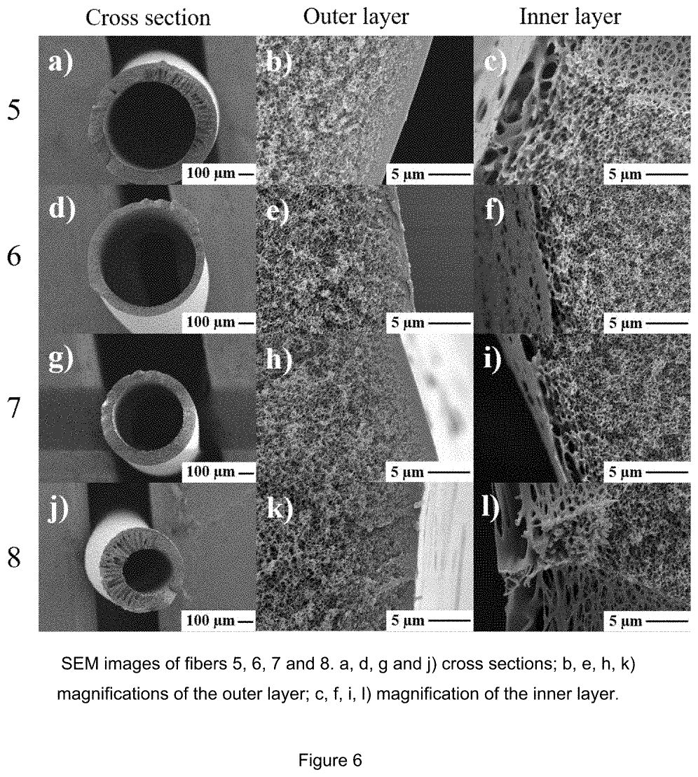

In regard now to the inside diameter of the fiber, the effect of the bore flow rate on the fiber inside diameter was also studied. The bore liquid and the fiber lumen could be expected to be closely associated with each other in terms of space. It is expected that in general, lower bore flow rate would lead to smaller inner diameter of the fibers. As expected, for fibers F1 and F2 the decrease of the bore flow rate from 0.9 mL/min to 0.4 mL/min, respectively, leads to a significant decrease of the inner diameter from 564 μm to 368 μm (FIG. 3, Table 2). In another comparison, when the bore flow rate was still further decreased for fibers F6 (0.4 mL/min), F7 (0.2 mL/min) and fiber F8 (0.1 mL/min) at a constant dope flow rate of 0.4 mL/min, the inner diameter of the fibers decreased considerably from about 369 μm (fiber F6) to 276 μm (fiber F7) and 191 μm (fiber F8); however, the wall thickness increased from 34 μm (fiber F6) to 72 μm (fiber F8) (FIG. 4, Table 2). In the case of fibers F14 and F15, as a consequence of the increase of the bore flow rate from 0.1 mL/min (F14) to 0.2 mL/min (F15), the inner diameter of fiber F15 increased by approximately 40 μm (FIG. 6, Table 2). Unfortunately, fiber F14 does not have reproducible desirable morphology because some segments of the fiber have inner and outer circumferences that are not perfectly circular. Fiber F15 has regular and reproducible morphology along all the length of the spun fibers. FIG. 13 shows a plot, for all of the experimental fibers, of measured fiber inside diameter as a function of bore flowrate only.

### Example 8: Correlation of Measured Inside Diameter to Volumetrically Calculated Inside Diameter

It is possible to perform a volumetric calculation expressing that the fiber inside diameter is represented by the volume of bore fluid dispensed and taking into consideration the stretching caused by the take-up wheel. For this purpose, the velocity of the exiting bore liquid was calculated using the known flowrate of the bore liquid and using diameter of the orifice that extrudes the bore liquid, which was 200 microns. It is possible to calculate the total volume of bore liquid flow, in a given amount of time, and divide that by the linear distance of the take-up wheel in the same given amount of time, and obtain a calculated cross-sectional area of the bore liquid and hence a calculation of the inside diameter of the fiber that is based on volumetric calculations and conservation of volume. In FIG. 14, this volumetrically calculated inside diameter is plotted as the horizontal axis. The vertical axis is the measured inside diameter of particular fibers. There is a consistent pattern except that the slope is not unity as would be expected (illustrated by the dashed line). It can be understood that the data in FIG. 14 includes variations in several of the other manufacturing parameters, which were varied in various ways and combinations during the 16 experiments.

The relevant formulas are:

Vbore=Qbore/Abore

Cross-sectional area of fiber lumen=Abore*Vbore/Vtakeup

Calculated inside diameter of fiber=sqrt(cross-sec area of fiber*4/π)

### Example 9: Effect of Shower Flowrate

In regard to the shower, eventually the shower joins the coagulation bath, and in embodiments of the invention the exterior of the forming fiber is exposed to the same composition in the shower and in the coagulation bath. In the present experiments, both the shower and the coagulation bath are pure water. All fibers were spun using a shower of ultra-pure water with flow rate of 0.3 mL/min. This flow rate was selected because it allowed us to obtain a regular dense outside layer without morphological irregularities. It is found (in other experimentation not reported here) that with higher flow rates of the shower, the morphology of the fibers is quite irregular, while with lower flow rates it is not possible to obtain a dense outer selective layer. The results illustrate the effects of many parameters of the spinning procedure (i.e., bore liquid composition, dope composition, dope and bore liquid flow rates, take-up wheel speed, air gap length and polymer dope concentration).

One way in which delamination/irregularity could be correlated with shower flowrate is as a function of the flowrate ratio Qdope/Qshower. In FIG. 15A, the data are plotted in his manner. It can be seen that delamination/irregularity occurs for Qdope/Qshower>1.2, and delamination/irregularity does not occur for Qdope/Qshower>1.2.

FIG. 15B illustrates a categorization of the fibers as irregular/delaminated or normal, correlated with a parameter that may be called the Speed Ratio. The Speed Ratio is the ratio of the linear velocity of the dope at the spinneret exit with the linear velocity of the shower at the spinneret exit. The Speed Ratio makes this comparison based on the possibility that velocity equilibration between bore and dope might not actually occur because the air gap is so short.

Vshower=Qshower/Ashower

Vdope=Qdope/Adope

Speed Ratio=Vshower/Vdope

where Qshower is the volumetric flowrate of the shower, Qdope is the volumetric flowrate of the dope, Ashower is the cross-sectional area of the annularly shaped shower channel, and Adope is the cross-sectional area of the annularly shaped dope channel.

It happens that for the spinneret used herein, the area of the dope channel Adope and the area of the shower channel Ashower are very close to equal to each other. Thus, the ordinates of the two plots FIG. 15A and FIG. 15B happen to be quite similar to each other in magnitude.

It can also be realized that, as discussed in an earlier Example, the current limited set of experimental data also shows a correlation between fiber external shape/delamination, as a function of the Stretch Ratio. The fibers are acceptable for a speed ratio greater than about 1.5 and are irregular/delaminated for a speed ratio less than about 1.5. It is possible that the data is not sufficiently detailed or varied to discern whether correlations with the Speed Ratio or the flowrate ratio Qdope/Qshower are perhaps just another way of representing the trend already exhibited by the Stretch Ratio.

### Example 10: Effect of Air Gap Length

In the set of 16 experimental conditions, 15 of the 16 experimental fibers were manufactured using an identical air gap of 0.6 cm. Only fiber F16 was manufactured using a different air gap, which was 1.2 cm. Thus, the ability to form conclusions about the effect of air gap is limited. Direct comparison of fiber F16 with other fibers cannot be made because more parameters than just the air gap were adjusted at the same time. Some comparison can be made to fiber F15, for which besides the shorter air gap, a lower dope flow rate (0.4 mL/min instead of 0.5 mL/min) was used. Despite having a relatively large dope flow rate, which is expected to increase the size of the fibers (especially the wall thickness), the dimensions of fiber F16 are slightly smaller compared to the dimensions of fiber F15 (FIG. 6, Table 2). It is believed that this small shrinking effect can be attributed to the longer air gap, which introduces an elongational stress due to gravity on the fiber that is emerging from the spinneret. In general, in embodiments of the invention, the length of the gap in which the shower liquid surrounds the dope can be a distance, between the spinneret exit and the coagulation bath, of about 80 to about 200 times a diameter of the shower channel.

### Example 11: Location and Thickness of Selective Layer

It is important that a fiber of embodiments of the invention to be used for hemodialysis applications should have a selective outer layer. This layer should hold back albumin and other blood proteins thus preventing their transport to the dialysate.

Formation of a tight selective layer is promoted by exposing the desired surface to a non-solvent or a liquid having a high concentration of a non-solvent. In this case the desired location of the selective layer is the outer surface of the hollow fiber. In these experiments the non-solvent is pure water and the water is applied both as a shower and as the coagulation bath. The shower surrounds the emerging dope between the spinneret exit and the coagulation bath. The shower then merges with the coagulation bath while the emerging fiber continues to be surrounded by the coagulation bath liquid.

In order to avoid the formation of a selective layer at the lumen side, we investigated the effect of the concentration of the organic solvent (NMP) in the bore liquid. It is believed that the presence of the organic solvent in the bore liquid in high concentrations slows down the phase separation process and, as a consequence, could contribute to obtaining a more open membrane structure at the lumen side. Three different NMP concentrations (50, 75 and 90 wt %) in the bore liquid were used (Table 1). The 50% NMP concentration was used only for two early experiments (fibers F1 and F2) in which it was found that a dense layer was formed on the luminal surface, in addition to the dense layer on the exterior surface. The dense inner (luminal) layer is considered undesirable for the present application. Therefore, the NMP concentration in the bore liquid was increased for all subsequent experiments. An NMP concentration of 75% was used for all of the later experiments, with the exception of Fiber 4, for which a 90% concentration was used. When the concentration of NMP has a higher value of either 75 wt % (fiber F3) or 90 wt % (fiber F4), an open lumen surface is obtained, while the layer on the outside surface of the fiber remains dense (FIG. 2).

In regard to the actual thickness of the dense selective layer on the outer surface of the fiber, using SEM photographs, we measured the thickness of the dense selective layer using the software ImageJ. ImageJ is an open source image processing program designed for scientific multidimensional images.

Table 2 shows the measured thicknesses of the outer selective layer for all of the fibers. It appears that the bore flow rate does not have any significant effect on the thickness of the selective layer on the exterior surface of the fiber. Actually, no significant difference is observed between fiber F1 (selective layer thickness 2.4±0.3 μm) and fiber F2 (selective layer thickness 2.6±0.4 μm), as well as among fiber F6 (selective layer thickness 1.3±0.4 μm), fiber F7 (selective layer thickness 1.3±0.0 μm) and fiber F8 (selective layer thickness 1.5±0.1 μm), and between fiber F14 (selective layer thickness 0.9±0.0 μm) and fiber F15 (selective layer thickness 0.7±0.1 μm).

It appears that there is some correlation between the thickness of the selective layer and the dope flowrate. The dope flow rates applied for the fabrication of fibers F3 (1 mL/min), fiber F5 (0.6 mL/min) and fiber F6 (0.4 mL/min), in decreasing order, correlates with a decreasing thickness of the selective layer also decreases due to slower dope flow rate (fiber F3: 6.9±0.2 μm, fiber F5:1.4±0.0 μm and fiber F6: 1.3±0.4 μm).

The thicknesses of the selective layers on the external surfaces of fiber F2 (2.6±0.6 μm) and fiber F4 (2.1±0.0 μm) are similar; however, the selective layer of fiber F3 is much thicker (6.9±0.2 μm) compared to those of fiber F2 and fiber F4 (which is a statistically significant difference per p<0.0001).

For fibers F15 and F16, three different SEM images of the selective layer of fiber F16 were used for the measurement. Three measurements for each fiber were taken (9 measurements in total). The average of those measurements is 0.6 microns. For the present application, it is considered useful for the thickness of the selective layer to be as small as possible, such as less than 1 micron, as long as the selective layer remains intact and mechanically robust. Along with the thickness of the selective layer, the porosity of the selective layer is a strong influence. It is believed that this contributes to the achievement of a membrane that has both a high permeability and a low Albumin Sieving Coefficient. A selective layer thickness of even less than this, such as <0.5 microns, is desirable to achieve high flux properties. For example, it appears that some commercial dialyzers (Buck and Gohl), whose selective layer in on the lumen surface, have a selective layer thickness of 0.3 to 0.5 microns.

It is possible, in embodiments of the invention, to have a selective layer or a dense layer on the luminal surface also. This occurred in fibers F1 and F2.

### Example 12: Presence or Absence of Elongated Macrovoids

Elongated macrovoids were observed completely or partially in 10 of the 16 experimental manufacturing conditions, although some of these 10 fibers exhibited delamination or irregular shape which is not preferred.

The fibers that present a spongy structure, without any macrovoids, are fibers F1-F4 and F6 and F7. Fibers that exhibit a partial pattern of macrovoids are fibers F5, F8, F11, F12, F13. The fibers that fully exhibit a macrovoid structure are fibers F9, F10, F13, F14, F15. There is somewhat of a pattern, although not a perfect pattern, that the spongy-structure fibers were manufactured using relatively larger values of dope flowrate or relatively larger values of combined flowrate of dope plus bore liquid. The fibers that exhibited macrovoids were manufactured using relatively smaller values of those parameters. This is illustrated in Table 2 and FIGS. 5-8.

In some of the SEM photographs, there can be observed to be macrovoids that are tapered or teardrop-shaped, pointing either in a radially inward direction or a radially outward direction. FIG. 16 contains various photographs illustrating details of macrovoids.

### Example 13: Characterization of the Preferred Fibers According to Various Parameters

After the just-described set of experiments involving 16 different fiber manufacturing conditions, it was decided to concentrate on more detailed characterization of a subset of those fibers. The selection of fibers for further evaluation was done on the basis of fiber dimensions, fiber morphology and transport properties. Fibers F15 and F16 showed desirable properties and these fibers were studied in the greatest detail. These represent a preferred embodiment of the invention.

A feature of fibers of an embodiment of the invention is the outer diameter of the fiber and the dimensional thickness of the selective layer. The selective layer can be visually observed in microphotographs such as Scanning Electron Microscope photographs of a cross-section of the fiber wall that has been cut perpendicular to the long direction of the fiber. The selective layer is on the outer surface of the fiber. Qualitatively, the selective layer appears to have pores that are relatively small and closely-packed, in contrast to the rest of the cross-section of the fiber, which has pores that are larger and more open (and sometimes also has macrovoids).

In general, and as described schematically in FIG. 2, a fiber of an embodiment of the invention may comprise a selective layer on the outside and, more interiorly, a supporting porous region, which may be called a supporting porous layer. Some or all of the supporting porous region may be spongy in nature, having a somewhat uniform distribution of pores that are larger than the pores of the selective layer. In some embodiments of the invention, a further feature within the supporting porous region may be the presence of elongated macrovoids that open or nearly open to the interior of the fiber. In such a fiber, the outer dense selective layer forms a continuous boundary that performs selective filtration as a function of the Molecular Weight of the solute. It is believed, although it is not wished to be limited to this explanation, that the presence of elongated macrovoids located between the outer dense layer and the lumen of the fiber, in fluid communication with the fiber lumen, provides a low-resistance flowpath for the passage of liquid therethrough, while the supporting porous region still provides adequate structural support for the selective layer. This combination of features further may contribute to the classification and performance of the membrane as high flux.

For an embodiment of the invention, cross-sectional photographs taken with a Scanning Electron Microscope are shown in FIG. 16. On the outside surface the fiber has a relatively thin layer of more-dense porous material, and in the remainder of the wall region it has a less-dense porous material. It is also found that in certain embodiments elongated macrovoids in communication with the lumen are formed. It is believed that the elongated macrovoids in communication with the lumen are helpful for achieving desired permeability because they provide a low-resistance flowpath for outside-in flow that has already passed through the selective layer, while at the same time providing structural support to the selective layer. As shown in the Figures, some of the elongated macrovoids may be tapered.

It is believed that the sieving characteristics of the fiber are determined primarily by the selective layer. It is believed that the presence of elongated macrovoids causes the flow resistance of the porous supporting region of the wall to be smaller (which is desirable for present purposes) than would be the case if the entire porous supporting region were uniformly porous. It is, however, desirable that the elongated macrovoids do not compromise the mechanical properties of the membrane. In our mechanical testing experiments we have seen that, even with the presence of elongated macrovoids, Fiber 16 has good mechanical properties.

### Example 14: Skin Thickness

We measured the thickness of the dense layer using the software ImageJ. ImageJ is an open source image processing program designed for scientific multidimensional images. Three different SEM images of the selective layer of fiber F16 were used for the measurement. Three measurements for each fiber were taken (9 measurements in total). The average of those measurements is 0.6±0.1 microns. The skin layer thickness of all of the experimental fibers is given in Table 2. In contrast, in Krause and Gohl, the thickness of the dense layer is estimated as 2-4 microns. Furthermore, in Krause and Gohl, the porous supporting region is spongy without having elongated macrovoids.

### Example 15: The Effect of Concentration of Polymer in the Dope on Fiber Dimensions

All of the fibers except for three fibers were spun using dope that had the composition PES 15 wt %, PVP 7 wt %, NMP 78 wt %. Three fibers (F14, F15 and F16) were spun using dope that contained a lower polymer concentration (PES 12 wt %, PVP 5.6 wt %, NMP 82.4 wt %). It can be noted that for both dope compositions, the relative concentration of PES was 2.14 times the concentration of PVP. Thus, throughout the experiments there was always the same ratio of PES/PVP, and the only difference between the two dope compositions was that in one the polymer overall was slightly more dilute than in the other. With lower polymer concentrations in the dope, it can be expected that some dimensions of the fibers may be smaller (especially having thinner walls), and/or the wall may be more porous, and the ultrafiltration coefficient (KUf) can be expected to be higher (which is often desirable in dialysis applications). To isolate this effect, the morphologies of fibers F14 and F15 can be compared to those of fibers F8 and F7, respectively. Fiber F14 was spun with the same parameters as fiber F8, and fiber F15 was spun with the same spinning parameters as fiber F7. The only differences are the polymer dope concentrations (Table 1). Even though fibers F14 and F8 do not have uniform structure, the average wall thickness of fiber F14 is approximately 20 μm thinner compared to that of fiber F8. It is believed that the lower polymer concentration in the dope used for the fabrication of fiber F15 leads to reduction of the inner diameter by about 30 μm, instead of to a decrease of the wall thickness. For these reasons, we cannot clearly conclude which dimensional parameter was most affected by the change of the polymer dope concentration, but it appears that in general, with other manufacturing parameters being held constant, reduction of the polymer concentration leads to a reduction of at least some of the fiber dimensions.

### Example 16: The Effect of Concentration of Polymer in the Dope on Thickness of the Selective Layer and on Permeability

It appears that decreasing the concentration of the polymer in the dope reduces the thickness of the selective layer. In the experiments, two different concentrations of polymer in organic solvent were used. For fibers F14 and F15, the dope was a more dilute solution of polymer in organic solvent, and the thickness of the selective layer of fiber F14 (0.9±0.1 μm) and fiber F15 (0.7±0.1 μm) was relatively thinner. For comparison, for fibers F7 and F8, the dope was a more concentrated solution of polymer in organic solvent, and the thickness of the selective layer was significantly greater as in fiber F8 (1.5±0.1 μm) and fiber F7 (1.3±0.0 μm).

### Example 17: Water Permeability

As a description of the flux or permeability for some of the fibers produced herein, the water transport properties of the fibers F3, F4, F8, F14, F15 and F16 are presented in Table 3. The property is expressed in one column as Ultrafiltration Coefficient (KUf) calculated extrapolated for a dialyzer having a surface area of 2 m² so it has units of (mL/(mmHg·h·2 m²)). In another column of Table 3, the permeability is presented in more universal units of mL/(mmHg·h m²).

TABLE 3

| | KUf | Permeability |
|---|---|---|
| Fiber | mL/(mmHg · h · 2 m²) | mL/(mmHg · h m²) |
| F3 | 19.6 ± 0.2 (n = 3) | 9.8 ± 0.1 (n = 3) |
| F4 | 476 ± 90 (n = 2) | 238 ± 45 (n = 2) |
| F8 | 22.6 ± 2.6 (n = 2) | 11.3 ± 1.3 (n = 2) |
| F14 | 62.2 ± 4.8 (n = 2) | 31.1 ± 2.4 (n = 2) |
| F15 | 25.1 ± 2.9 (n = 3) | 12.5 ± 1.4 (n = 3) |
| F16 | 31.6 ± 6.4 (n = 3) | 15.8 ± 3.2 (n = 3) |

It is possible to compare experimental fibers produced with two different compositions of bore liquid with no other parameters being varied, by comparing fiber F3 (NMP concentration in the bore liquid of 75%) to fiber F4 (NMP concentration in the bore liquid from 90%), it is found that the increase of NMP concentration in the bore liquid from 75% (for fiber F3) to 90% (for fiber F4) leads to an increase of permeability from 9.8±0.1 mL/(mmHg·h m²) (for fiber F3) to 238±45 mL/(mmHg·h m²) (for fiber F4), which is a significant increase. The selective layer of fiber F3 (6.9±0.2 μm) is much thicker compared to that of fiber F4 (2.1±0.0 μm). It is believed that the presence of NMP in the bore liquid slows down the process of phase separation, thus contributing to the formation of fibers that are more open on their interior surface. This suggests that the supporting porous layer also plays a role in the overall water permeability of the fiber wall.

The effect of the polymer dope concentration on the membrane transport properties can be observed by comparing fiber F14 and fiber F8. Fiber F14 was spun with the same spinning parameters as for fiber F8, except for using lower polymer dope concentration (Table 1 and Table 2). Fiber F14 presents significantly higher permeability compared to fiber F8 (31 vs. 11 mL/(mmHg·h m²), respectively and this result is consistent with the fact that fiber F14 has a thinner selective layer and a thinner wall thickness. A thinner selective layer and thinner wall are generally associated with lower mass transport resistance, or greater permeability.

### Example 18: Bovine Serum Albumin (BSA) Filtration Experiments

Albumin filtration experiments were performed for fibers F3, F4, F8, F15, F16. The albumin sieving coefficients (calculated as described elsewhere herein) are shown in Table 4. Values are shown as mean±standard deviation.

TABLE 4

| Fiber | Bovine Serum Albumin Sieving Coefficient |
|---|---|
| F3 | 0.00 ± 0.00 (n = 2) |
| F4 | 0.98 ± 0.00 (n = 2) |
| F8 | 0.02 ± 0.01 (n = 2) |
| F15 | 0.02 ± 0.00 (n = 3) |
| F16 | 0.09 ± 0.01 (n = 2) |

The BSA SC filtration results are consistent with the measured permeability and with the morphological characteristics reported elsewhere herein. In fact, the Albumin Sieving Coefficients of fibers F3, F8 and F15 are low, which is consistent with their relatively low permeability values (see Table 3). In the case of fiber F4, the high Albumin Sieving Coefficient of fiber F4 (allowing passage of albumin) can be easily related to the fact that it also has an extremely high permeability. Due to this high albumin leakage, fiber F4 is not considered desirable. Fiber F16 has slightly higher albumin Sieving Coefficient compared to fibers F3, F8 and F15, but still would be acceptable for hemodialysis applications. This finding is consistent with the permeability results, in that the permeability of fiber F16 is slightly higher compared to those of fibers F3, F8 and F15.

### Example 19: Vitamin B12 Filtration

Vitamin B12 (MW 1355 Da) is a neutral organic solute that is often used to estimate the sieving properties of dialysis membranes. Fiber F16 was selected for further transport studies with vitamin B12. For all fibers F16 studied (n=3), the Sieving Coefficient for Vitamin B12 was equal to 1.00±0.00. This indicates that fiber F16 is completely permeable to Vitamin B12.

### Example 20: Creatinine Transport Experiment

Referring now to FIG. 17, there are shown experimental results (n=3) of the creatinine transport from human plasma across the fiber F16. For these experiments we used a fixed flow rate for plasma and for dialysate (plasma flow rate: 10 mL/min in the space outside the fibers, and dialysate flow rate of 1 mL/min in the intraluminal space) in order to achieve zero transmembrane pressure across the membrane. The membrane removes approximately 2000 mg/m² in 4 hours and 4700 mg/m2 of creatinine in 24 hours. During the first 4 hours the kinetics of removal follows a linear trend, but after that the removal becomes slower, which may be an artificial effect due to the limited amount of fluids that were recirculated during the experiment.

As a comparison, the commercial Fresenius F8HPS fiber (which is an Inside-Out fiber having its selective layer at the lumen) can remove, via inside-out filtration, approximately 3400 mg/m² of creatinine from Phosphate Buffered Saline in 4 hours, but with the removal rate decreasing after that.

It is possible that at low flow rates, retained blood proteins adsorb onto surfaces of the membrane, instead of being well dispersed in the flowing blood. The “layer” of blood proteins deposited on surfaces of the membrane can decrease the filtration ability of the membrane and can cause the measured concentration of the proteins in the flowing blood to be lower than it would be if that adsorption were not occurring.

### Example 21: Sieving Performance as a Function of Molecular Weight

The experimentation has included measurement of the passage through the membrane of the following substances: albumin (having a Molecular Weight of approximately 67 KDa); vitamin B12 (having a Molecular Weight of approximately 1.4 KDa); creatinine (having a Molecular Weight of approximately 113 Da); and of course water.

From this experimentation, we have found that fiber F16 retains albumin (having a Molecular Weight of approximately 67 KDa), quantified as having an albumin sieving coefficient as given elsewhere herein, and this fiber is completely permeable to vitamin B12 (having a Molecular Weight of approximately 1.4 KDa). Of course, if the membrane is completely permeable to Vitamin B12, it also would be completely permeable to the even lower molecular weight molecules such as creatinine. From this data it can be inferred that the Molecular Weight Cut Off of the fiber F16 is less than 67 kDa, but it is believed that the cutoff is probably close to 67 kDa, closer to 67 kDa than it is to the molecular weight of Vitamin B12.

The experiments involving creatinine were performed in a situation of zero trans-membrane pressure (TMP). The other experiments were performed by applying to the fiber a trans-membrane pressure (TMP) of 1 Bar in a dead-end configuration. This TMP is much higher than the pressure that is typically applied in clinical uses (which is usually in the range of 50-100 mbar).

### Example 22: Surface Chemistry Characterization

The fibers described herein were made using polyethersulfone (PES) as the membrane forming polymer. PES, on one hand, has excellent filtering characteristics, thermal stability, mechanical strength, and chemical inertness, and it can withstand all typical sterilization techniques. On the other hand, its hydrophobic nature favors adhesion of proteins on the membrane, which not only affects membrane performance but also can trigger a series of other reactions such as activation of the coagulation cascade, blood clotting, complement and fibrinolysis reactions. Therefore, blending of PES with a hydrophilic additive offers improvement in terms of reducing of protein adhesion and improving hemocompatibility. In this work, polyvinylpyrrolidone (PVP) was used as the hydrophilic additive to PES. The distribution of PVP on the membrane surfaces of fiber F16 was studied by means of ATR-FTIR and XPS. For the present application involving Outside-In Filtration, the outer surface of the fiber is the surface that contacts the blood, and therefore it is desirable if the outer surface contains a sufficient concentration of PVP.

FIG. 18A compares the ATR-FTIR spectra of the outer surface of fiber F16 and of the outer surface of fiber F8HPS (Fresenius). FIG. 18A also shows the spectra of pure powder of PES and PVP. The peak at 1677 cm⁻¹, corresponding to the carbonyl groups of PVP, has noticeably higher intensity for fiber F16 in comparison to the intensity for fiber F8HPS. This indicates higher concentration of PVP at the outer surface of the fiber F16 in comparison to the concentration of PVP at the outer surface of the F8HPS fiber.

Using another technique as presented in FIG. 18B, XPS measurements were performed on the outer and inner surfaces of fiber F16 and on the outer and inner surfaces of the fiber F8HPS. These measurements provide the elemental molar percentages of several elements. The nitrogen concentration is highlighted because in this polymer system the PVP is the only substance that contains any nitrogen. The results suggest that the PVP is well distributed within fiber F16. The nitrogen concentration on the outer surface of fiber F16 is similar to the concentration that is found on the inner surface of the fiber F8HPS. The F8HPS fiber is a fiber that is widely used commercially for hemodialysis, because of its good hemocompatibility property (on its luminal surface).

### Example 23: Mechanical Tests

Referring now to FIGS. 19A-19C, mechanical testing was performed using 13 samples of the fiber F16, and, for comparison, using 5 samples of the commercial fiber F8HPS. The F8HPS fiber is an inside-out fiber, having its selective layer on the lumen surface, and is used in conventional dialyzers. Fiber samples having a length >5 cm were clamped at both ends and were pulled at constant elongation velocity of 50 mm/min until they broke. Ultimate tensile strength, Young's Modulus and elongation at ultimate strength were measured.

FIG. 19A compares the Young's Modulus (E) (ratio of stress/strain) of fiber F16 to that of the commercially available fiber F8HPS. In regard to Young's Modulus, there is no statistically significant difference between the two fibers concerning elastic deformation. FIG. 19B shows the maximum strength at breakage. FIG. 19C shows the maximum elongation at breakage. As shown in FIG. 19B and FIG. 19C, the maximum strength and maximum elongation before breakage are lower for the fiber of an embodiment of the invention, compared to the commercial fiber, and the difference does have statistical significance at p<0.05.

It is believed that the Young's Modulus value is the most relevant parameter for hemodialysis applications and, based on this, we can conclude that the fiber F16 can withstand the pressure and typical stress encountered in hemodialysis, and it is believed that the mechanical strength of fiber F16 and its elongation at breakage are adequate.

### Example 24: Reproducibility Study

Reproducibility tests were performed using fibers F15 and F16. The reproducibility tests involved producing two additional batches of each of these fibers after the initial batch, under the same conditions as the initial batch. Fibers F15 and F16 were chosen because the properties of those fibers were considered optimum. The morphology and transport properties of the fibers from the various batches were compared among each other and with the initial batch. Specifically, SEM imaging, water transport and albumin filtration experiments were performed on the two additional batches.

The batch for the initial investigation is referred to as batch 1 and is presented elsewhere herein such as in Table 1 and Table 2. Two additional batches (referred to as batches 2 and 3) were spun and were compared to the first batch. FIGS. 20 and 21 present typical SEM images of the cross-sections, inner and outer layers of the three different batches of fibers F15 and F16. For fiber F15, FIG. 20 shows SEM images of batches 1, 2 and 3. Images a, d, g) are cross-sections; b, e, h) are magnifications of the outer layer; c, f, i) are magnifications of the inner layer. For fiber F16, FIG. 21 shows SEM images of batches 1, 2 and 3. Images a, d, g) are cross-sections; b, e, h) are magnifications of the outer layer; c, f, i) are magnifications of the inner layer. Table 5 presents dimensions and performance details of the three batches of fibers F15 and F16. Specifically, Table 5 presents outside diameter; inside diameter; wall thickness; ultrafiltration coefficient values (KUF) and sieving coefficients (SC) of bovine serum albumin (BSA). The latter two quantities are expressed as average±standard deviation.

TABLE 5

| | F15 | | | F16 | | |
|---|---|---|---|---|---|---|
| | Batch 1 | Batch 2 | Batch 3 | Batch 1 | Batch 2 | Batch 3 |
| Outside Diameter (μm) | 338 | 334 | 347 | 322 | 345 | 345 |
| Inside Diameter (μm) | 243 | 248 | 257 | 236 | 256 | 258 |
| Wall (μm) | 48 | 42 | 45 | 42 | 40 | 46 |

| | | |
|---|---|---|
| KUf (mL/(h · mmHg)) for · 2m² | 34 ± 11 (n = 5) | 30 ± 7 (n = 5) |
| BSA SC | 0.02 ± 0.01 (n = 5) | 0.05 ± 0.04 (n = 4) |

For fibers prepared in batch 2, for both fibers F15 and fibers F16, the lumen of the fiber is not very well centered and, for this reason, one side of the wall is thicker than the other. This deviation is possibly caused by lack of alignment of the spinneret with the coagulation bath. The images of the cross sections of the various batches of fiber F15 are very similar, with elongated macrovoids and comparable inner diameter and wall thickness. Moreover, the same morphology of the outer and inner layers can be observed. The same conclusions regarding morphological reproducibility can be made also for fiber F16. For fibers F15, the averaged KUF for the three batches of fibers F15 was 34±11 mL/(h·mmHg) for·2 m². For fibers F16, the averaged KUF was 30±7 mL/(h·mmHg) for·2 m². For all batches of fibers F15 and fibers F16, the BSA SC (Bovine Serum Albumin Sieving Coefficient) is very small.

In general, comparing fiber F15 and F16, the structures of fibers F15 and fibers F16 are comparable, their transport properties are similar, and both fibers could be suitable for Outside-In Filtration. It is believed that, due to the fact that the air gap used for the production of fiber F16 is slightly longer (and easier to apply for upscaling), there would be some preference for producing fiber F16.

### Additional Comments

Although embodiments have been disclosed in connection with fibers for dialysis, it is also possible for similar constructs to be used for other applications. In general, any combination of disclosed features, components and methods described herein is possible. Steps of a method can be performed in any order that is physically possible.

Embodiments of the invention use a dope that comprises polymer dissolved in an organic solvent. Embodiments of the invention use the polymer family of polyethersulfone and polyvinylpyrrolidone, but other polymers could also be used.

All cited references are incorporated by reference herein.

Although embodiments have been disclosed, it is not desired to be limited thereby. Rather, the scope should be determined only by the appended claims.

1. A porous hollow fiber, said fiber comprising:
(a) a tubular body comprising a wall region and defining a lumen surrounded by said wall region, and
(b) said wall region comprising an outer surface, an inner surface, said wall region extending in a radial direction from said outer surface to said inner surface, wherein said inner surface defines said lumen, said outer surface and said inner surface are generally concentric with each other, and said wall region and said lumen extend in an axial direction; wherein:
(i) said wall region comprises a porous fiber composition containing a mixture of a polysulfone polymer and a hydrophilic polymer;
(ii) said wall region has a wall thickness from said inner surface to said outer surface of about 20 μm to about 40 μm and wherein said porous hollow fiber has an outside diameter of about 200 μm to about 300 μm;
(iii) said wall region comprises a bulk layer and a selective layer, wherein the bulk layer supports the selective layer and the selective layer is located radially outwardly from the bulk layer, and said selective layer is selective for exclusion of passage of albumin therethrough so that said porous hollow fiber has an albumin sieving coefficient of less than approximately 0.01;
(iv) said wall region comprises a plurality of radially extending elongated macrovoids located in said bulk layer; and
(v) said porous hollow fiber has a permeability for water through said wall region of at least approximately 6 mL/(h·mmHg·m²).

2. The porous hollow fiber of claim 1, wherein said plurality of radially extending elongated macrovoids have a void width dimension, which is a dimension transverse to said radial direction, that is at least five times as large as an average pore size of pores within said bulk layer and excluding said plurality of elongated macrovoids.

3. The porous hollow fiber of claim 1, wherein said plurality of radially extending elongated macrovoids have a radially extending dimension that is at least 50% of a wall thickness of said wall region.

4. The porous hollow fiber of claim 1, wherein said plurality of radially extending elongated macrovoids have a shape having a radially extending dimension that is at least twice a void width dimension, which is a dimension transverse to said radial direction and measured at a midpoint along said plurality of radially extending elongated macrovoids.

5. The porous hollow fiber of claim 1, wherein a portion of the bulk layer is located between said plurality of radially extending elongated macrovoids and said selective layer.

6. The porous hollow fiber of claim 1, wherein at least a portion of said plurality of radially extending elongated macrovoids are open to said lumen.

7. The porous hollow fiber of claim 1, wherein said wall region further comprises an inner selective layer located between said plurality of radially extending elongated macrovoids and said lumen.

8. The porous hollow fiber of claim 1, wherein said plurality of radially extending elongated macrovoids have a shape comprising a radial dimension of about 10 μm to about 30 μm and a dimension transverse to the radial direction and measured at a midpoint along said plurality of radially extending elongated macrovoids of about 2 μm to about 10 μm.

9. The porous hollow fiber of claim 1, wherein said wall region comprises a circumferential density of said plurality of radially extending elongated macrovoids of at least 50 macrovoids per circumference in a cross section of said porous hollow fiber.

10. The porous hollow fiber of claim 1, wherein said wall region comprises a spacing of said plurality of radially extending elongated macrovoids, from a center of one of said macrovoids to a center of a neighboring macrovoid, in a range of approximately 10 to 20 μm.

11. The porous hollow porous hollow fiber of claim 1, wherein said selective layer has a thickness of less than 1 μm.

12. The porous hollow fiber of claim 1, wherein said selective layer comprises pores sufficiently small so that said selective layer provides a molecular weight cutoff of less than about 66 KDaltons.

13. The porous hollow fiber of claim 1, wherein said selective layer has an average pore size of less than about 5 nanometers.

14. The porous hollow fiber of claim 1, wherein said porous hollow fiber has a blood albumin retention coefficient of greater than approximately 0.99 when measured for flow in a direction from said outer surface to said inner surface.

15. The porous hollow fiber of claim 1, wherein said outer surface has a root-mean-square surface roughness of less than approximately 20 nanometers.

16. The porous hollow fiber of claim 1, wherein said polysulfone polymer comprises at least one of polysulfone, polyethersulfone, and polyarylethersulfone.

17. The porous hollow fiber of claim 1, wherein said polysulfone polymer comprises polyethersulfone, and said porous fiber composition comprises a weight ratio of said polyethersulfone to said hydrophilic polymer of about 4:1 to about 1:2.

18. The porous hollow fiber of claim 1, wherein said polysulfone polymer comprises polyethersulfone, and said porous fiber composition comprises a weight ratio of said polyethersulfone to said hydrophilic polymer of about 3:1 to about 1:1.

19. The porous hollow fiber of claim 1, wherein said polysulfone polymer comprises polyethersulfone and said hydrophilic polymer comprises polyvinylpyrrolidone.

20. The porous hollow fiber of claim 19, wherein said selective layer comprises a concentration of said polyvinylpyrrolidone that is greater than a concentration of said polyvinylpyrrolidone in said bulk layer.

21. The porous hollow fiber of claim 19, wherein said porous fiber composition comprises at least 40 wt. % said polyethersulfone and at least 20 wt. % said polyvinylpyrrolidone.

22. The porous hollow fiber of claim 1, wherein said outer surface has a polyvinylpyrrolidone concentration of at least 3.6%.

23. The porous hollow fiber of claim 1, wherein said hydrophilic polymer comprises polyethylene glycol.

24. The porous hollow fiber of claim 19, wherein said hydrophilic polymer further comprises polyethylene glycol.

25. The porous hollow fiber of claim 16, wherein said polyethersulfone comprises a derivative of polyethersulfone.

26. The porous hollow fiber of claim 1, wherein said porous hollow fiber has a beta-2-microglobulin sieving coefficient of at least approximately 0.7.

27. A dialyzer cartridge comprising a plurality of the fibers of claim 1, further comprising:
a housing having a housing interior including a housing midsection interior region, a housing blood supply port, a housing blood discharge port;
a first end barrier that joins with said fibers at first ends of said fibers and joins with said housing interior of said housing and bounds a first end plenum and separates said first end plenum from said housing midsection interior region;
a second end barrier that joins with said fibers at second ends of said fibers and joins with said housing interior and bounds a second end plenum and separates said second end plenum from said housing midsection interior region,
wherein a blood flow compartment comprises an inter fiber space defined by said fiber exteriors and an interior housing surface along said housing midsection interior region, said housing blood supply port and said housing blood discharge port, said inter fiber space, said housing supply port, and said housing discharge port being in fluid communication with each other, and
wherein a fluid flow compartment comprises said first end plenum, said fiber interiors, and said second end plenum, said first end plenum, said fiber interiors, and said second end plenum being in fluid communication with each other.

28. A porous hollow fiber, said fiber comprising:
(a) a tubular body comprising a wall region and defining a lumen surrounded by said wall region, and
(b) said wall region comprising an outer surface, an inner surface, said wall region extending in a radial direction from said outer surface to said inner surface, wherein said inner surface defines said lumen, said outer surface and said inner surface are generally concentric with each other, and said wall region and said lumen extend in an axial direction; wherein:
(i) said wall region comprises a porous fiber composition containing a mixture of a polysulfone polymer and a hydrophilic polymer;
(ii) said wall region comprises a bulk layer and a selective layer, wherein the bulk layer supports the selective layer and the selective layer is located radially outwardly from the bulk layer, and said selective layer is selective for exclusion of passage of albumin therethrough so that said porous hollow fiber has an albumin sieving coefficient of less than approximately 0.01;
(iii) said wall region comprises a plurality of radially extending elongated macrovoids located in said bulk layer; and
(iv) said porous hollow fiber has a permeability for water through said wall region of at least approximately 6 mL/(h·mmHg·m²).

29. A method of producing a hollow fiber, said method comprising:
forming an emergent fiber from a triple concentric spinneret having a bore liquid channel, a dope channel annularly surrounding said bore liquid channel, and a shower channel annularly surrounding said dope channel, by flowing a bore liquid through said bore liquid channel, flowing a dope liquid through said dope channel, and flowing a shower liquid through said shower channel; and
stretching said emergent fiber as said emergent fiber passes through a coagulation bath while being pulled at a take-up velocity,
wherein said triple concentric spinneret, said bore liquid, said dope, said shower liquid, and said coagulation bath are provided at temperatures within a 5 degrees C. range of each other,
wherein said bore liquid comprises a fiber forming polymer composition containing an organic solvent, and
wherein said shower liquid and said coagulation bath comprise respective higher concentrations of a non-solvent compared to said bore liquid.

30. The method of claim 28, wherein said stretching comprises stretching said emergent fiber to a fiber stretch ratio of between 1 and 4, wherein a combined volumetric flowrate is calculated as a volumetric flowrate of said bore liquid plus a volumetric flowrate of said dope liquid, a geometric extrusion velocity is calculated as said combined volumetric flowrate divided by a cross-sectional area of said dope channel plus a cross-sectional area of said bore channel of said spinneret, and said fiber stretch ratio is defined as said take-up velocity divided by said geometric extrusion velocity.
